SEQUENCE LISTING (48 sequences)

SEQ ID NO: 1 - DNA, 3218 bp, Saccharomyces cerevisiae
aaataggaatgtgataccttctattgcatgcaaagatagtgtaggaggcgctgctattgccaaagacttttgagaccgcttgctgtttcattatagttgaggagttctcgaagacgagaaattagcagttttcggtgtttagtaatcgcgctagcatgctaggacaatttaactgcaaaattttgatacgatagtgatagtaaatggaaggtaaaaataacatagacctatcaataagcaatgtctctcagaataaaagcacttgatgcatcagtggttaacaaaattgctgcaggtgagatcataatatcccccgtaaatgctctcaaagaaatgatggagaattccatcgatgcgaatgctacaatgattgatattctagtcaaggaaggaggaattaaggtacttcaaataacagataacggatctggaattaataaagcagacctgccaatcttatgtgagcgattcacgacgtccaaattacaaaaattcgaagatttgagtcagattcaaacgtatggattccgaggagaagctttagccagtatctcacatgtggcaagagtcacagtaacgacaaaagttaaagaagacagatgtgcatggagagtttcatatgcagaaggtaagatgttggaaagccccaaacctgttgctggaaaagacggtaccacgatcctagttgaagacctttttttcaatattccttctagattaagggccttgaggtcccataatgatgaatactctaaaatattagatgttgtcgggcgatacgccattcattccaaggacattggcttttcttgtaaaaagttcggagactctaattattctttatcagttaaaccttcatatacagtccaggataggattaggactgtgttcaataaatctgtggcttcgaatttaattacttttcatatcagcaaagtagaagatttaaacctggaaagcgttgatggaaaggtgtgtaatttgaatttcatatccaaaaagtccatttcattaatttttttcattaataatagactagtgacatgtgatcttctaagaagagctttgaacagcgtttactccaattatctgccaaagggcttcagaccttttatttatttgggaattgttatagatccggcggctgttgatgttaacgttcacccgacaaagagagaggttcgtttcctgagccaagatgagatcatagagaaaatcgccaatcaattgcacgccgaattatctgccattgatacttcacgtactttcaaggcttcttcaatttcaacaaacaagccagagtcattgataccatttaatgacaccatagaaagtgataggaataggaagagtctccgacaagcccaagtggtagagaattcatatacgacagccaatagtcaactaaggaaagcgaaaagacaagagaataaactagtcagaatagatgcttcacaagctaaaattacgtcatttttatcctcaagtcaacagttcaactttgaaggatcgtctacaaagcgacaactgagtgaacccaaggtaacaaatgtaagccactcccaagaggcagaaaagctgacactaaatgaaagcgaacaaccgcgtgatgccaatacaatcaatgataatgacttgaaggatcaacctaagaagaaacaaaagttgggggattataaagttccaagcattgccgatgacgaaaagaatgcactcccgatttcaaaagacgggtatattagagtacctaaggagcgagttaatgttaatcttacgagtatcaagaaattgcgtgaaaaagtagatgattcgatacatcgagaactaacagacatttttgcaaatttgaattacgttggggttgtagatgaggaaagaagattagccgctattcagcatgacttaaagctttttttaatagattacggatctgtgtgctatgagctattctatcagattggtttgacagacttcgcaaactttggtaagataaacctacagagtacaaatgtgtcagatgatatagttttgtataatctcctatcagaatttgacgagttaaatgacgatgcttccaaagaaaaaataattagtaaaatatgggacatgagcagtatgctaaatgagtactattccatagaattggtgaatgatggtctagataatgacttaaagtctgtgaagctaaaatctctaccactacttttaaaaggctacattccatctctggtcaagttaccattttttatatatcgcctgggtaaagaagttgattgggaggatgaacaagagtgtctagatggtattttaagagagattgcattactctatatacctgatatggttccgaaagtcgatacactcgatgcatcgttgtcagaagacgaaaaagcccagtttataaatagaaaggaacacatatcctcattactagaacacgttctcttcccttgtatcaaacgaaggttcctggcccctagacacattctcaaggatgtcgtggaaatagccaaccttccagatctatacaaagtttttgagaggtgttaactttaaaacgttttggctgtaataccaaagtttttgtttatttcctgagtgtgattgtgtttcatttgaaagtgtatgccctttcctttaacgattcatccgcgagatttcaaaggatatgaaatatggttgcagttaggaaagtatgtcagaaatgtatattcggattgaaactcttctaatagttctgaagtcacttggttccgtattgttttcgtcctcttcctcaagcaacgattcttgtctaagcttattcaacggtaccaaagacccgagtccttttatgagagaaaacatttcatcatttttcaactcaattatcttaatatcattttgtagtattttgaaaacaggatggtaaaacgaatcacctgaatctagaagctgtaccttgtcccataaaagttttaatttactgagcctttcggtcaagtaaactagtttatctagttttgaaccgaatattgtgggcagatttgcagtaagttcagttagatctactaaaagttgtttgacagcagccgattccacaaaaatttggtaaaaggagatgaaagagacctcgcgcgtaatggtttgcatcaccatcggatgtctgttgaaaaactcactttttgcatggaagttattaacaataagactaatgattaccttagaataatgtataa

SEQ ID NO: 2 - protein, 769 aa, Saccharomyces cerevisiae
MSLRIKALDASVVNKIAAGEIIISPVNALKEMMENSIDANATMIDILVKEGGIKVLQITDNGSGINKADLPILCERFTTSKLQKFEDLSQIQTYGFRGEALASISHVARVTVTTKVKEDRCAWRVSYAEGKMLESPKPVAGKDGTTILVEDLFFNIPSRLRALRSHNDEYSKILDVVGRYAIHSKDIGFSCKKFGDSNYSLSVKPSYTVQDRIRTVFNKSVASNLITFHISKVEDLNLESVDGKVCNLNFISKKSISLIFFINNRLVTCDLLRRALNSVYSNYLPKGFRPFIYLGIVIDPAAVDVNVHPTKREVRFLSQDEIIEKIANQLHAELSAIDTSRTFKASSISTNKPESLIPFNDTIESDRNRKSLRQAQVVENSYTTANSQLRKAKRQENKLVRIDASQAKITSFLSSSQQFNFEGSSTKRQLSEPKVTNVSHSQEAEKLTLNESEQPRDANTINDNDLKDQPKKKQKLGDYKVPSIADDEKNALPISKDGYIRVPKERVNVNLTSIKKLREKVDDSIHRELTDIFANLNYVGVVDEERRLAAIQHDLKLFLIDYGSVCYELFYQIGLTDFANFGKINLQSTNVSDDIVLYNLLSEFDELNDDASKEKIISKIWDMSSMLNEYYSIELVNDGLDNDLKSVKLKSLPLLLKGYIPSLVKLPFFIYRLGKEVDWEDEQECLDGILREIALLYIPDMVPKVDTLDASLSEDEKAQFINRKEHISSLLEHVLFPCIKRRFLAPRHILKDVVEIANLPDLYKVFERC

SEQ ID NO: 3 - protein, 859 aa, Mus musculus
MEQTEGVSTECAKAIKPIDGKSVHQICSGQVILSLSTAVKELIENSVDAGATTIDLRLKDYGVDLIEVSDNGCGVEEENFEGLALKHHTSKIQEFADLTQVETFGFRGEALSSLCALSDVTISTCHGSASVGTRLVFDHNGKITQKTPYPRPKGTTVSVQHLFYTLPVRYKEFQRNIKKEYSKMVQVLQAYCIISAGVRVSCTNQLGQGKRHAVVCTSGTSGMKENIGSVFGQKQLQSLIPFVQLPPSDAVCEEYGLSTSGRHKTFSTFRASFHSARTAPGGVQQTGSFSSSIRGPVTQQRSLSLSMRFYHMYNRHQYPFVVLNVSVDSECVDINVTPDKRQILLQEEKLLLAVLKTSLIGMFDSDANKLNVNQQPLLDVEGNLVKLHTAELEKPVPGKQDNSPSLKSTADEKRVASISRLREAFSLHPTKEIKSRGPETAELTRSFPSEKRGVLSSYPSDVISYRGLRGSQDKLVSPTDSPGDCMDREKIEKDSGLSSTSAGSEEEFSTPEVASSFSSDYNVSSLEDRPSQETINCGDLDCRPPGTGQSLKPEDHGYQCKALPLARLSPTNAKRFKTEERPSNVNISQRLPGPQSTSAAEVDVAIKMNKRIVLLEFSLSSLAKRMKQLQHLKAQNKHELSYRKFRAKICPGENQAAEDELRKEISKSMFAEMEILGQFNLGFIVTKLKEDLFLVDQHAADEKYNFEMLQQHTVLQAQRLITPQTLNLTAVNEAVLIENLEIFRKNGFDFVIDEDAPVTERAKLISLPTSKNWTFGPQDIDELIFMLSDSPGVMCRPSRVRQMFASRACRKSVMIGTALNASEMKKLITHMGEMDHPWNCPHGRPTMRHVANLDVISQN

SEQ ID NO: 4 - DNA, 3056 bp, Mus musculus
gaattccggtgaaggtcctgaagaatttccagattcctgagtatcattggaggagacagataacctgtcgtcaggtaacgatggtgtatatgcaacagaaatgggtgttcctggagacgcgtcttttcccgagagcggcaccgcaactctcccgcggtgactgtgactggaggagtcctgcatccatggagcaaaccgaaggcgtgagtacagaatgtgctaaggccatcaagcctattgatgggaagtcagtccatcaaatttgttctgggcaggtgatactcagtttaagcaccgctgtgaaggagttgatagaaaatagtgtagatgctggtgctactactattgatctaaggcttaaagactatggggtggacctcattgaagtttcagacaatggatgtggggtagaagaagaaaactttgaaggtctagctctgaaacatcacacatctaagattcaagagtttgccgacctcacgcaggttgaaactttcggctttcggggggaagctctgagctctctgtgtgcactaagtgatgtcactatatctacctgccacgggtctgcaagcgttgggactcgactggtgtttgaccataatgggaaaatcacccagaaaactccctacccccgacctaaaggaaccacagtcagtgtgcagcacttattttatacactacccgtgcgttacaaagagtttcagaggaacattaaaaaggagtattccaaaatggtgcaggtcttacaggcgtactgtatcatctcagcaggcgtccgtgtaagctgcactaatcagctcggacaggggaagcggcacgctgtggtgtgcacaagcggcacgtctggcatgaaggaaaatatcgggtctgtgtttggccagaagcagttgcaaagcctcattccttttgttcagctgccccctagtgacgctgtgtgtgaagagtacggcctgagcacttcaggacgccacaaaaccttttctacgtttcgggcttcatttcacagtgcacgcacggcgccgggaggagtgcaacagacaggcagtttttcttcatcaatcagaggccctgtgacccagcaaaggtctctaagcttgtcaatgaggttttatcacatgtataaccggcatcagtacccatttgtcgtccttaacgtttccgttgactcagaatgtgtggatattaatgtaactccagataaaaggcaaattctactacaagaagagaagctattgctggccgttttaaagacctccttgataggaatgtttgacagtgatgcaaacaagcttaatgtcaaccagcagccactgctagatgttgaaggtaacttagtaaagctgcatactgcagaactagaaaagcctgtgccaggaaagcaagataactctccttcactgaagagcacagcagacgagaaaagggtagcatccatctccaggctgagagaggccttttctcttcatcctactaaagagatcaagtctaggggtccagagactgctgaactgacacggagttttccaagtgagaaaaggggcgtgttatcctcttatccttcagacgtcatctcttacagaggcctccgtggctcgcaggacaaattggtgagtcccacggacagccctggtgactgtatggacagagagaaaatagaaaaagactcagggctcagcagcacctcagctggctctgaggaagagttcagcaccccagaagtggccagtagctttagcagtgactataacgtgagctccctagaagacagaccttctcaggaaaccataaactgtggtgacctggactgccgtcctccaggtacaggacagtccttgaagccagaagaccatggatatcaatgcaaagctctacctctagctcgtctgtcacccacaaatgccaagcgcttcaagacagaggaaagaccctcaaatgtcaacatttctcaaagattgcctggtcctcagagcacctcagcagctgaggtcgatgtagccataaaaatgaataagagaatcgtgctcctcgagttctctctgagttctctagctaagcgaatgaagcagttacagcacctaaaggcgcagaacaaacatgaactgagttacagaaaatttagggccaagatttgccctggagaaaaccaagcagcagaagatgaactcagaaaagagattagtaaatcgatgtttgcagagatggagatcttgggtcagtttaacctgggatttatagtaaccaaactgaaagaggacctcttcctggtggaccagcatgctgcggatgagaagtacaactttgagatgctgcagcagcacacggtgctccaggcgcagaggctcatcacaccccagactctgaacttaactgctgtcaatgaagctgtactgatagaaaatctggaaatattcagaaagaatggctttgactttgtcattgatgaggatgctccagtcactgaaagggctaaattgatttccttaccaactagtaaaaactggacctttggaccccaagatatagatgaactgatctttatgttaagtgacagccctggggtcatgtgccggccctcacgagtcagacagatgtttgcttccagagcctgtcggaagtcagtgatgattggaacggcgctcaatgcgagcgagatgaagaagctcatcacccacatgggtgagatggaccacccctggaactgcccccacggcaggccaaccatgaggcacgttgccaatctggatgtcatctctcagaactgacacaccccttgtagcatagagtttattacagattgttcggtttgcaaagagaaggttttaagtaatctgattatcgttgtacaaaaattagcatgctgctttaatgtactggatccatttaaaagcagtgttaaggcaggcatgatggagtgttcctctagctcagctacttgggtgatccggtgggagctcatgtgagcccaggactttgagaccactccgagccacattcatgagactcaattcaaggacaaaaaaaaaaagatatttttgaagccttttaaaaaaaaa

SEQ ID NO: 5 - protein, 932 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQKPSHLGQGTTVTALRLFKNLPVRKQFYSTAKKCKDEIKKIQDLLMSFGILKPDLRIVFVHNKAVIWQKSRVSDHKMALMSVLGTAVMNNMESFQYHSEESQIYLSGFLPKCDADHSFTSLSTPERSFIFINSRPVHQKDILKLIRHHYNLKCLKESTRLYPVFFLKIDVPTADVDVNLTPDKSQVLLQNKESVLIALENLMTTCYGPLPSTNSYENNKTDVSAADIVLSKTAETDVLFNKVESSGKNYSNVDTSVIPFQNDMHNDESGKNTDDCLNHQISIGDFGYGHCSSEISNIDKNTKNAFQDISMSNVSWENSQTEYSKTCFISSVKHTQSENGNKDHIDESGENEEEAGLENSSEISADEWSRGNILKNSVGENIEPVKILVPEKSLPCKVSNNNYPIPEQMNLNEDSCNKKSNVIDNKSGKVTAYDLLSNRVIKKPMSASALFVQDHRPQFLIENPKTSLEDATLQIEELWKTLSEEEKLKYEEKATKDLERYNSQMKRAIEQESQMSLKDGRKKIKPTSAWNLAQKHKLKTSLSNQPKLDELLQSQIEKRRSQNIKMVQIPFSMKNLKINFKKQNKVDLEEKDEPCLIHNLRFPDAWLMTSKTEVMLLNPYRVEEALLFKRLLENHKLPAEPLEKPIMLTESLFNGSHYLDVLYKMTADDQRYSGSTYLSDPRLTANGFKIKLIPGVSITENYLEIEGMANCLPFYGVADLKEILNAILNRNAKEVYECRPRKVISYLEGEAVRLSRQLPMYLSKEDIQDIIYRMKHQFGNEIKECVHGRPFFHHLTYLPETT

SEQ ID NO: 6 - DNA, 2771 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttcggatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctgaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactgaccgtagtcactgtatggaataattggttttatcgcagatttttatgttttgaaagacagagtcttcactaaccttttttgttttaaaatgaaacctgctacttaaaaaaaatacacatcacacccatttaaaagtgatcttgagaaccttttcaaacc

SEQ ID NO: 7 - protein, 932 aa, Homo sapiens; identical to SEQ ID NO: 5

SEQ ID NO: 8 - DNA, 3063 bp, Homo sapiens
ggcacgagtggctgcttgcggctagtggatggtaattgcctgcctcgcgctagcagcaagctgctctgttaaaagcgaaaatgaaacaattgcctgcggcaacagttcgactcctttcaagttctcagatcatcacttcggtggtcagtgttgtaaaagagcttattgaaaactccttggatgctggtgccacaagcgtagatgttaaactggagaactatggatttgataaaattgaggtgcgagataacggggagggtatcaaggctgttgatgcacctgtaatggcaatgaagtactacacctcaaaaataaatagtcatgaagatcttgaaaatttgacaacttacggttttcgtggagaagccttggggtcaatttgttgtatagctgaggttttaattacaacaagaacggctgctgataattttagcacccagtatgttttagatggcagtggccacatactttctcagaaaccttcacatcttggtcaaggtacaactgtaactgctttaagattatttaagaatctacctgtaagaaagcagttttactcaactgcaaaaaaatgtaaagatgaaataaaaaagatccaagatctcctcatgagctttggtatccttaaacctgacttaaggattgtctttgtacataacaaggcagttatttggcagaaaagcagagtatcagatcacaagatggctctcatgtcagttctggggactgctgttatgaacaatatggaatcctttcagtaccactctgaagaatctcagatttatctcagtggatttcttccaaagtgtgatgcagaccactctttcactagtctttcaacaccagaaagaagtttcatcttcataaacagtcgaccagtacatcaaaaagatatcttaaagttaatccgacatcattacaatctgaaatgcctaaaggaatctactcgtttgtatcctgttttctttctgaaaatcgatgttcctacagctgatgttgatgtaaatttaacaccagataaaagccaagtattattacaaaataaggaatctgttttaattgctcttgaaaatctgatgacgacttgttatggaccattacctagtacaaattcttatgaaaataataaaacagatgtttccgcagctgacatcgttcttagtaaaacagcagaaacagatgtgctttttaataaagtggaatcatctggaaagaattattcaaatgttgatacttcagtcattccattccaaaatgatatgcataatgatgaatctggaaaaaacactgatgattgtttaaatcaccagataagtattggtgactttggttatggtcattgtagtagtgaaatttctaacattgataaaaacactaagaatgcatttcaggacatttcaatgagtaatgtatcatgggagaactctcagacggaatatagtaaaacttgttttataagttccgttaagcacacccagtcagaaaatggcaataaagaccatatagatgagagtggggaaaatgaggaagaagcaggtcttgaaaactcttcggaaatttctgcagatgagtggagcaggggaaatatacttaaaaattcagtgggagagaatattgaacctgtgaaaattttagtgcctgaaaaaagtttaccatgtaaagtaagtaataataattatccaatccctgaacaaatgaatcttaatgaagattcatgtaacaaaaaatcaaatgtaatagataataaatctggaaaagttacagcttatgatttacttagcaatcgagtaatcaagaaacccatgtcagcaagtgctctttttgttcaagatcatcgtcctcagtttctcatagaaaatcctaagactagtttagaggatgcaacactacaaattgaagaactgtggaagacattgagtgaagaggaaaaactgaaatatgaagagaaggctactaaagacttggaacgatacaatagtcaaatgaagagagccattgaacaggagtcacaaatgtcactaaaagatggcagaaaaaagataaaacccaccagcgcatggaatttggcccagaagcacaagttaaaaacctcattatctaatcaaccaaaacttgatgaactccttcagtcccaaattgaaaaaagaaggagtcaaaatattaaaatggtacagatccccttttctatgaaaaacttaaaaataaattttaagaaacaaaacaaagttgacttagaagagaaggatgaaccttgcttgatccacaatctcaggtttcctgatgcatggctaatgacatccaaaacagaggtaatgttattaaatccatatagagtagaagaagccctgctatttaaaagacttcttgagaatcataaacttcctgcagagccactggaaaagccaattatgttaacagagagtctttttaatggatctcattatttagacgttttatataaaatgacagcagatgaccaaagatacagtggatcaacttacctgtctgatcctcgtcttacagcgaatggtttcaagataaaattgataccaggagtttcaattactgaaaattacttggaaatagaaggaatggctaattgtctcccattctatggagtagcagatttaaaagaaattcttaatgctatattaaacagaaatgcaaaggaagtttatgaatgtagacctcgcaaagtgataagttatttagagggagaagcagtgcgtctatccagacaattacccatgtacttatcaaaagaggacatccaagacattatctacagaatgaagcaccagtttggaaatgaaattaaagagtgtgttcatggtcgcccattttttcatcatttaacctatcttccagaaactacatgattaaatatgtttaagaagattagttaccattgaaattggttctgtcataaaacagcatgagtctggttttaaattatctttgtattatgtgtcacatggttattttttaaatgaggattcactgacttgtttttatattgaaaaaagttccacgtattgtagaaaacgtaaataaactaataac

SEQ ID NO: 9 - protein, 934 aa, Homo sapiens [sequence omitted]

SEQ ID NO: 10 - DNA, 3145 bp, Homo sapiens
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 11 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 12 - DNA, 2484 bp, Homo sapiens
cttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacata

SEQ ID NO: 13 - protein, 133 aa, Homo sapiens
MKQLPAATVRLLSSSQIITSVVSVVKELIENSLDAGATSVDVKLENYGFDKIEVRDNGEGIKAVDAPVMAMKYYTSKINSHEDLENLTTYGFRGEALGSICCIAEVLITTRTAADNFSTQYVLDGSGHILSQK

SEQ ID NO: 14 - DNA, 426 bp, Homo sapiens
cgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaacttga

SEQ ID NO: 15 - protein, 1360 aa, Homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 16 - DNA, 4264 bp, Homo sapiens
atttcccgccagcaggagccgcgcggtagatgcggtgcttttaggagctccgtccgacagaacggttgggccttgccggctgtcggtatgtcgcgacagagcaccctgtacagcttcttccccaagtctccggcgctgagtgatgccaacaaggcctcggccagggcctcacgcgaaggcggccgtgccgccgctgcccccggggcctctccttccccaggcggggatgcggcctggagcgaggctgggcctgggcccaggcccttggcgcgatccgcgtcaccgcccaaggcgaagaacctcaacggagggctgcggagatcggtagcgcctgctgcccccaccagttgtgacttctcaccaggagatttggtttgggccaagatggagggttacccctggtggccttgtctggtttacaaccacccctttgatggaacattcatccgcgagaaagggaaatcagtccgtgttcatgtacagttttttgatgacagcccaacaaggggctgggttagcaaaaggcttttaaagccatatacaggttcaaaatcaaaggaagcccagaagggaggtcatttttacagtgcaaagcctgaaatactgagagcaatgcaacgtgcagatgaagccttaaataaagacaagattaagaggcttgaattggcagtttgtgatgagccctcagagccagaagaggaagaagagatggaggtaggcacaacttacgtaacagataagagtgaagaagataatgaaattgagagtgaagaggaagtacagcctaagacacaaggatctaggcgaagtagccgccaaataaaaaaacgaagggtcatatcagattctgagagtgacattggtggctctgatgtggaatttaagccagacactaaggaggaaggaagcagtgatgaaataagcagtggagtgggggatagtgagagtgaaggcctgaacagccctgtcaaagttgctcgaaagcggaagagaatggtgactggaaatggctctcttaaaaggaaaagctctaggaaggaaacgccctcagccaccaaacaagcaactagcatttcatcagaaaccaagaatactttgagagctttctctgcccctcaaaattctgaatcccaagcccacgttagtggaggtggtgatgacagtagtcgccctactgtttggtatcatgaaactttagaatggcttaaggaggaaaagagaagagatgagcacaggaggaggcctgatcaccccgattttgatgcatctacactctatgtgcctgaggatttcctcaattcttgtactcctgggatgaggaagtggtggcagattaagtctcagaactttgatcttgtcatctgttacaaggtggggaaattttatgagctgtaccacatggatgctcttattggagtcagtgaactggggctggtattcatgaaaggcaactgggcccattctggctttcctgaaattgcatttggccgttattcagattccctggtgcagaagggctataaagtagcacgagtggaacagactgagactccagaaatgatggaggcacgatgtagaaagatggcacatatatccaagtatgatagagtggtgaggagggagatctgtaggatcattaccaagggtacacagacttacagtgtgctggaaggtgatccctctgagaactacagtaagtatcttcttagcctcaaagaaaaagaggaagattcttctggccatactcgtgcatatggtgtgtgctttgttgatacttcactgggaaagtttttcataggtcagttttcagatgatcgccattgttcgagatttaggactctagtggcacactatcccccagtacaagttttatttgaaaaaggaaatctctcaaaggaaactaaaacaattctaaagagttcattgtcctgttctcttcaggaaggtctgatacccggctcccagttttgggatgcatccaaaactttgagaactctccttgaggaagaatattttagggaaaagctaagtgatggcattggggtgatgttaccccaggtgcttaaaggtatgacttcagagtctgattccattgggttgacaccaggagagaaaagtgaattggccctctctgctctaggtggttgtgtcttctacctcaaaaaatgccttattgatcaggagcttttatcaatggctaattttgaagaatatattcccttggattctgacacagtcagcactacaagatctggtgctatcttcaccaaagcctatcaacgaatggtgctagatgcagtgacattaaacaacttggagatttttctgaatggaacaaatggttctactgaaggaaccctactagagagggttgatacttgccatactccttttggtaagcggctcctaaagcaatggctttgtgccccactctgtaaccattatgctattaatgatcgtctagatgccatagaagacctcatggttgtgcctgacaaaatctccgaagttgtagagcttctaaagaagcttccagatcttgagaggctactcagtaaaattcataatgttgggtctcccctgaagagtcagaaccacccagacagcagggctataatgtatgaagaaactacatacagcaagaagaagattattgattttctttctgctctggaaggattcaaagtaatgtgtaaaattatagggatcatggaagaagttgctgatggttttaagtctaaaatccttaagcaggtcatctctctgcagacaaaaaatcctgaaggtcgttttcctgatttgactgtagaattgaaccgatgggatacagcctttgaccatgaaaaggctcgaaagactggacttattactcccaaagcaggctttgactctgattatgaccaagctcttgctgacataagagaaaatgaacagagcctcctggaatacctagagaaacagcgcaacagaattggctgtaggaccatagtctattgggggattggtaggaaccgttaccagctggaaattcctgagaatttcaccactcgcaatttgccagaagaatacgagttgaaatctaccaagaagggctgtaaacgatactggaccaaaactattgaaaagaagttggctaatctcataaatgctgaagaacggagggatgtatcattgaaggactgcatgcggcgactgttctataactttgataaaaattacaaggactggcagtctgctgtagagtgtatcgcagtgttggatgttttactgtgcctggctaactatagtcgagggggtgatggtcctatgtgtcgcccagtaattctgttgccggaagataccccccccttcttagagcttaaaggatcacgccatccttgcattacgaagactttttttggagatgattttattcctaatgacattctaataggctgtgaggaagaggagcaggaaaatggcaaagcctattgtgtgcttgttactggaccaaatatggggggcaagtctacgcttatgagacaggctggcttattagctgtaatggcccagatgggttgttacgtccctgctgaagtgtgcaggctcacaccaattgatagagtgtttactagacttggtgcctcagacagaataatgtcaggtgaaagtacattttttgttgaattaagtgaaactgccagcatactcatgcatgcaacagcacattctctggtgcttgtggatgaattaggaagaggtactgcaacatttgatgggacggcaatagcaaatgcagttgttaaagaacttgctgagactataaaatgtcgtacattattttcaactcactaccattcattagtagaagattattctcaaaatgttgctgtgcgcctaggacatatggcatgcatggtagaaaatgaatgtgaagaccccagccaggagactattacgttcctctataaattcattaagggagcttgtcctaaaagctatggctttaatgcagcaaggcttgctaatctcccagaggaagttattcaaaagggacatagaaaagcaagagaatttgagaagatgaatcagtcactacgattatttcgggaagtttgcctggctagtgaaaggtcaactgtagatgctgaagctgtccataaattgctgactttgattaaggaattatagactgactacattggaagctttgagttgacttctgaccaaaggtggtaaattcagacaacattatgatctaataaactttattttttaaaaatga

SEQ ID NO: 17 - DNA, 1408 bp, Homo sapiens
ggcgctcctacctgcaagtggctagtgccaagtgctgggccgccgctcctgccgtgcatgttggggagccagtacatgcaggtgggctccacacggagaggggcgcagacccggtgacagggctttacctggtacatcggcatggcgcaaccaaagcaagagagggtggcgcgtgccagacaccaacggtcggaaaccgccagacaccaacggtcggaaaccgccaagacaccaacgctcggaaaccgccagacaccaacgctcggaaaccgccagacaccaaggctcggaatccacgccaggccacgacggagggcgactacctcccttctgaccctgctgctggcgttcggaaaaaacgcagtccggtgtgctctgattggtccaggctctttgacgtcacggactcgacctttgacagagccactaggcgaaaaggagagacgggaagtattttttccgccccgcccggaaagggtggagcacaacgtcgaaagcagccgttgggagcccaggaggcggggcgcctgtgggagccgtggagggaactttcccagtccccgaggcggatccggtgttgcatccttggagcgagctgagaactcgagtacagaacctgctaaggccatcaaacctattgatcggaagtcagtccatcagatttgctctgggccggtggtaccgagtctaaggccgaatgcggtgaaggagttagtagaaaacagtctggatgctggtgccactaatgttgatctaaagcttaaggactatggagtggatctcattgaagtttcaggcaatggatgtggggtagaagaagaaaacttcgaaggctttactctgaaacatcacacatgtaagattcaagagtttgccgacctaactcaggtggaaacttttggctttcggggggaagctctgagctcactttgtgcactgagtgatgtcaccatttctacctgccgtgtatcagcgaaggttgggactcgactggtgtttgatcactatgggaaaatcatccagaaaaccccctacccccgccccagagggatgacagtcagcgtgaagcagttattttctacgctacctgtgcaccataaagaatttcaaaggaatattaagaagaaacgtgcctgcttccccttcgccttctgccgtgattgtcagtttcctgaggcctccccagccatgcttcctgtacagcctgtagaactgactcctagaagtaccccaccccacccctgctccttggaggacaacgtgatcactgtattcagctctgtcaagaatggtccaggttcttctagatgatctgcacaaatggttcctctcctccttcctgatgtctgccattagcattggaataaagttcctgctgaaaatccaaaaaaaaaaaaaaaaaaaaaaa

SEQ ID NO: 18 - protein, 389 aa, Homo sapiens
MAQPKQERVARARHQRSETARHQRSETAKTPTLGNRQTPTLGNRQTPRLGIHARPRRRATTSLLTLLLAFGKNAVRCALIGPGSLTSRTRPLTEPLGEKERREVFFPPRPERVEHNVESSRWEPRRRGACGSRGGNFPSPRGGSGVASLERAENSSTEPAKAIKPIDRKSVHQICSGPVVPSLRPNAVKELVENSLDAGATNVDLKLKDYGVDLIEVSGNGCGVEEENFEGFTLKHHTCKIQEFADLTQVETFGFRGEALSSLCALSDVTISTCRVSAKVGTRLVFDHYGKIIQKTPYPRPRGMTVSVKQLFSTLPVHHKEFQRNIKKKRACFPFAFCRDCQFPEASPAMLPVQPVELTPRSTPPHPCSLEDNVITVFSSVKNGPGSSR

SEQ ID NO: 19 - DNA, 795 bp, Homo sapiens
atgtgtccttggcggcctagactaggccgtcgctgtatggtgagccccagggaggcggatctgggcccccagaaggacacccgcctggatttgccccgtagcccggcccgggcccctcgggagcagaacagccttggtgaggtggacaggaggggacctcgcgagcagacgcgcgcgccagcgacagcagccccgccccggcctctcgggagccggggggcagaggctgcggagccccaggagggtctatcagccacagtctctgcatgtttccaagagcaacaggaaatgaacacattgcaggggccagtgtcattcaaagatgtggctgtggatttcacccaggaggagtggcggcaactggaccctgatgagaagatagcatacggggatgtgatgttggagaactacagccatctagtttctgtggggtatgattatcaccaagccaaacatcatcatggagtggaggtgaaggaagtggagcagggagaggagccgtggataatggaaggtgaatttccatgtcaacatagtccagaacctgctaaggccatcaaacctattgatcggaagtcagtccatcagatttgctctgggccagtggtactgagtctaagcactgcagtgaaggagttagtagaaaacagtctggatgctggtgccactaatattgatctaaagcttaaggactatggagtggatctcattgaagtttcagacaatggatgtggggtagaagaagaaaactttgaaggcttaatctctttcagctctgaaacatcacacatgtaa

SEQ ID NO: 20 - protein, 264 aa, Homo sapiens
MCPWRPRLGRRCMVSPREADLGPQKDTRLDLPRSPARAPREQNSLGEVDRRGPREQTRAPATAAPPRPLGSRGAEAAEPQEGLSATVSACFQEQQEMNTLQGPVSFKDVAVDFTQEEWRQLDPDEKIAYGDVMLENYSHLVSVGYDYHQAKHHHGVEVKEVEQGEEPWIMEGEFPCQHSPEPAKAIKPIDRKSVHQICSGPVVLSLSTAVKELVENSLDAGATNIDLKLKDYGVDLIEVSDNGCGVEEENFEGLISFSSETSHM

SEQ ID NO: 21 - DNA, 1445 bp, Homo sapiens
tttttttttttgatgttctccagtgcctcagtggcagcagaactggccctgtatcaggccgctaccgccactccatgaccaacctccctgcatacccccccccccagcacccctcccacaggaccgcttctgtgtttgggacccaccaggcctttgcaccatacaacaaaccctcactctccggggcccggtctgcgcccaggctgaacaccacgaacgcctgggacgcagctcctccttccctggggagccagcccctctaccgctccagcctctcccacctgggaccgcagcacctgcccccaggatcctccacctccggtgcagtcagtgcctccctccccagcggtccctcaagcagcccaggcgagcgtccctgccactgtgcccatgcagatgccaagccagcagagtcagcaggcgctcgctggagcgacccgaagccagagcagagcagagcaggtcataaaactacacggaagagctgaaagtgcccccagatgaggactgcatcatctgcatggagaagctgtccgcagcgtctggatacagcgatgtgactgacagcaaggcaatggggcccctggctgtgggctgcctcaccaagtgcagccacgccttccacctgctgtgcctcctggccatgtactgcaacggcaataagggccctgagcaccccaatcccggaaagccgttcactgccagagggtttcccgccagtgctaccttccagacaacgccagggccgcaagcctccaggggcttccagaacccggagacactggctgacattccggcctccccacagctgctgaccgatggccactacatgacgctgcccgtgtctccggaccagctgccctgtgacgaccccatggcgggcagcggaggcgcccccgtgctgcgggtgggccatgaccacggctgccaccagcagccacgtatctgcaacgcgcccctccctggccctggaccctatcgtacagaacctgctaaggccatcaaacctattgatcggaagtcagtccatcagatttgctctgggccagtggtactgagtctaagcactgcagtgaaggagttagtagaaaacagtctggatgctggtgccactaatattgatctaaagcttaaggactatggaatggatctcattgaagtttcaggcaatggatgtggggtagaagaagaaaacttcgaaggcttaatgatgtcaccatttctacctgccacgtctcggcgaaggttgggactcgactggtgtttgatcacgatgggaaaatcatccagaagaccccctacccccaccccagagggaccacagtcagcgtgaagcagttattttctacgctacctgtgcgccataaggaatttcaaaggaatattaagaagaaacatgctgcttccccttcgccttctgccgtgattgtcagttttaaccggaa

SEQ ID NO: 22 - protein, 270 aa, Homo sapiens
MEKLSAASGYSDVTDSKAMGPLAVGCLTKCSHAFHLLCLLAMYCNGNKGPEHPNPGKPFTARGFPASATFQTTPGPQASRGFQNPETLADIPASPQLLTDGHYMTLPVSPDQLPCDDPMAGSGGAPVLRVGHDHGCHQQPRICNAPLPGPGPYRTEPAKAIKPIDRKSVHQICSGPVVLSLSTAVKELVENSLDAGATNIDLKLKDYGMDLIEVSGNGCGVEEENFEGLMMSPFLPATSRRRLGLDWCLITMGKSSRRPPTPTPEGPQSA

SEQ ID NO: 23 - protein, 16 aa, Homo sapiens
AVKELVENSLDAGATN

SEQ ID NO: 24 - protein, 48 aa, Homo sapiens
LRPNAVKELVENSLDAGATNVDLKLKDYGVDLIEVSGNGCGVEEENFE

SEQ ID NO: 25 - protein, 47 aa, Homo sapiens
LSTAVKELVENSLDAGATNIDLKLKDYGVDLIEVSDNGCGVEEENFE

SEQ ID NO: 26 - protein, 50 aa, Homo sapiens
LRQVLSNLLDNAIKYTPEGGEITVSLERDGDHLEITVEDNGPGIPEEDLE

SEQ ID NO: 27 - DNA, 22 bp, artificial
  Oligonucleotide primer
ggacgagaagtataacttcgag

SEQ ID NO: 28 - DNA, 21 bp, Artificial
  Oligonucleotide primer
catctcgcttgtgttaagagc

SEQ ID NO: 29 - DNA, 19 bp, Artificial
  Oligonucleotide primer
ggcgcaaccaaagcaagag

SEQ ID NO: 30 - DNA, 19 bp, Artificial
  Oligonucleotide primer
actgcgttttttccgaacg

SEQ ID NO: 31 - DNA, 19 bp, Artificial
  Oligonucleotide primer
atgttggagaactacagcc

SEQ ID NO: 32 - DNA, 19 bp, Artificial
  Oligonucleotide primer
cactccatagtccttaagc

SEQ ID NO: 33 - DNA, 19 bp, Artificial
  Oligonucleotide primer
gggaatgggtcagaaggac

SEQ ID NO: 34 - DNA, 20 bp, Artificial
  Oligonucleotide primer
tttcacggttggccttaggg

SEQ ID NO: 35 - DNA, 21 bp, Artificial
  Oligonucleotide primer
tgactacttttgacttcagcc

SEQ ID NO: 36 - DNA, 22 bp, Artificial
  Oligonucleotide primer
aaccattcaacatttttaaccc

SEQ ID NO: 37 - DNA, 21 bp, Artificial
  Oligonucleotide primer
attaacttcctacaccacaac

SEQ ID NO: 38 - DNA, 19 bp, Artificial
  Oligonucleotide primer
gtagagcaagaccaccttg

SEQ ID NO: 39 - DNA, 20 bp, Artificial
  Oligonucleotide primer
acattgctggaagttctggc

SEQ ID NO: 40 - DNA, 20 bp, Artificial
  Oligonucleotide primer
cctttctgacttggatacca

SEQ ID NO: 41 - protein, 20 aa, Homo sapiens
MAQPKQERVARARHQRSETA

SEQ ID NO: 42 - protein, 20 aa, Homo sapiens
LEDNVITVFSSVKNGPGSSR

SEQ ID NO: 43 - protein, 20 aa, Homo sapiens
RPRLGRRCMVSPRARAPREQ

SEQ ID NO: 44 - protein, 20 aa, Homo sapiens
GVEEENFEGLISFSSETSHM

SEQ ID NO: 45 - DNA, 30 bp, Artificial
  Oligonucleotide primer
acgcatatggagcgagctgagagctcgagt

SEQ ID NO: 46 - DNA, 75 bp, Artificial
  Oligonucleotide primer
gaattcttatcacgtagaatcgagaccgaggagagggttagggataggcttaccagttccaaccttcgccgatgc

SEQ ID NO: 47 - DNA, 27 bp, Artificial
  Oligonucleotide primer
acgcatatgtgtccttggcggcctaga

SEQ ID NO: 48 - DNA, 75 bp, Artificial
  Oligonucleotide primer
gaattcttattacgtagaatcgagaccgaggagagggttagggataggcttacccatgtgtgatgtttcagagct

(19) United States
(12) Patent Application Publication
(10) Pub. No.: US 2003/0170895 A1
(43) Pub. Date: Sep. 11, 2003

(54) Methods of making hypermutable cells using PMSR homologs

(76) Inventors: Luigi Grasso, Bala Cynwyd, PA (US); Nicholas C. Nicolaides, Boothwyn, PA (US); Philip M. Sass, Audubon, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/371,634

(22) Filed: Feb. 21, 2003

Related U.S. Application Data
(60) Provisional application No. 60/358,578, filed on Feb. 21, 2002.

(51) Int. Cl. C12N 15/85; C12N 5/06

(52) U.S. Cl. 435/455; 435/325

(57) ABSTRACT

Methods of making cells hypermutable are disclosed using PMS2 homologs that have a common sequence motif. The PMS2 homologs of the invention have ATPase-like motifs and are at least about 90% identical to PMS2-134. Methods of generating mutant libraries and using the PMS2 homologs in diagnostic and therapeutic applications for cancer are also disclosed.

### CROSS-REFERENCE TO RELATED APPLICATIONS

This application claims the benefit of U.S. Provisional Application No. 60/358,578, filed Feb. 21, 2002, the disclosure of which is incorporated by reference herein in its entirety.

### FIELD OF THE INVENTION

The invention is related to the area of mismatch repair genes. In particular it is related to the field of generating hypermutable cells using dominant negative mismatch repair genes wherein the proteins encoded by the mismatch repair gene comprise a consensus sequence for an ATPase.

### BACKGROUND OF THE INVENTION

Within the past four years, the genetic cause of the Hereditary Nonpolyposis Colorectal Cancer Syndrome (HNPCC), also known as Lynch syndrome II, has been ascertained for the majority of kindred's affected with the disease (Liu, B. et al. (1996) Nat. Med. 2:169-174). The molecular basis of HNPCC involves genetic instability resulting from defective mismatch repair (MMR). To date, six genes have been identified in humans that encode for proteins and appear to participate in the MMR process, including the mutS homologs GTBP, hMSH2, hMSH3 and the mutL homologs hMLH1, hPMSI, and hPMS2 (Bronner, C. E. et al. (1994) Nature 368:258-261; Fishel, R. et al. (1993) Cell 7:1027-1038; Leach, F. S. et al. (1993) Cell 75:1215-1225; Nicolaides, N. C., et al. (1994) Nature 371:75-80; Nicolaides, N. C. et al. (1996) Genomics 31:395-397; Palombo, F. et al. (1995) Science 268:1912-1914; Papadopoulos, N. et al. (1994) Science 263:1625-1629). Mutations or epigenetic changes affecting the function of these genes have been reported for all of the homologs listed above in tumor tissues exhibiting microsatellite instability (MI), a type of genomic instability that results from “slippage mutations” in mono-, di, or tri-nucleotide repeats due to MMR deficiency (Jiricny, J., and M. Nystrom-Lahti (2000) Curr. Opin. Genet. Dev. 10:157-161; Perucho, M. (1996) Biol. Chem. 377:675-684; Strand, M. et al. (1993) Nature 365:274-276). While germline mutations in all of these genes have been identified in HNPCC kindreds (Bronner, C. E. et al. (1994) Nature 368:258-261; Leach, F. S. et al. (1993) Cell 75:1215-1225; Liu, B. et al. (1996) Nat. Med. 2:169-174; Nicolaides, N. C. et al. (1994) Nature 371:75-80; Papadopoulos, N. et al. (1994) Science 263:1625-1629), many examples exist where tumor types exhibiting MI lack mutations in any of the known MMR genes, suggesting the presence of additional genes that are involved in the MMR process (personal observation; Nagy, M. et al. (2000) Leukemia 14:2142-2148; Peltomaki P. (2001) Hum. Mol. Genet. 10:735-740; Wang Y. et al. (2001) Int. J. Cancer 93:353-360). In addition to its occurrence in virtually all tumors arising in HNPCC patients, MI is also found in a subset of sporadic tumors with distinctive molecular and phenotypic properties originating from many different tissue types, suggesting a role for an expanded involvement of defective MMR in other cancer types (Nagy, M. et al. (2000) Leukemia 14:2142-2148; Peltomaki P. (2001) Hum. Mol. Genet. 10:735-740; Wang Y. et al. (2001) Int. J. Cancer 93:353-360; Starostik P. et al. (2000) Am. J. Pathol. 157:1129-1136; Chen Y. et al. (2001) Cancer Res. 61:4112-4121).

Though the mutator defect that arises from the MMR deficiency can affect any DNA sequence, microsatellite sequences are particularly sensitive to MMR abnormalities (Modrich, P. (1994) Science 266:1959-1960). Microsatellite instability (MI) is therefore a useful indicator of defective MMR. In addition to its occurrence in virtually all tumors arising in HNPCC patients, MI is found in a small fraction of sporadic tumors with distinctive molecular and phenotypic properties (Perucho, M. (1996) Biol. Chem. 377:675-684).

HNPCC is inherited in an autosomal dominant fashion, so that the normal cells of affected family members contain one mutant allele of the relevant MMR gene (inherited from an affected parent) and one wild-type allele (inherited from the unaffected parent). During the early stages of tumor development, however, the wild-type allele is inactivated through a somatic mutation, leaving the cell with no functional MMR gene and resulting in a profound defect in MMR activity. Because a somatic mutation in addition to a germ-line mutation is required to generate defective MMR in the tumor cells, this mechanism is generally referred to as one involving two hits, analogous to the biallelic inactivation of tumor suppressor genes that initiate other hereditary cancers (Leach, F. S. et al. (1993) Cell 75:1215-1225; Liu, B. et al. (1996) Nat. Med. 2:169-174; Parsons, R. et al. (1993) Cell 75:1227-1236). In line with this two-hit mechanism, the non-neoplastic cells of HNPCC patients generally retain near normal levels of MMR activity due to the presence of the wild-type allele.

Genetic studies have unequivocally shown that inactivation of mismatch repair (MMR) genes, including PMS2, results in genetic instability and tumorigenesis in human and rodent tissues. In the majority of cases, inactivation of both alleles of a particular MMR gene are required to completely knockout a component of the MMR spell check system, a process that is similar to the “two-hit” hypothesis for inactivation of tumor suppressor alleles. Independent studies focused on screening for mutated MMR genes in normal and neoplastic tissues have confirmed the two hit hypothesis except for 2 cases where only a single mutated allele of a MMR gene was found associated in tumors. This allele is a PMS2 gene containing a nonsense mutation at codon 134, which results in a truncated polypeptide that encodes for a 133 amino acid protein capable of eliciting a dominant negative effect on the MMR activity of the cell. This hypothesis was confirmed by subsequent studies demonstrating the ability of the PMS134 protein to cause a dominant negative effect on the MMR activity of an otherwise MMR proficient mammalian cell.

The truncated domain of PMS134 is highly homologous to the coding region of PMSR2 and PMSR3 proteins, sharing an identity of greater than 90% at the protein level. However, PMSR2 and PMSR3 do not appear to be expressed in normal tissues and have not been shown to be associated with HNPCC.

The ability to alter the signal transduction pathways by manipulation of a gene products function, either by over-expression of the wild type protein or a fragment thereof, or by introduction of mutations into specific protein domains of the protein, the so-called dominant-negative inhibitory mutant, were described over a decade in the yeast system Saccharomyces cerevisiae by Herskowitz (1987) Nature 329 (6136):219-222). It has been demonstrated that over-expression of wild type gene products can result in a similar, dominant-negative inhibitory phenotype due most likely to the “saturating-out” of a factor, such as a protein, that is present at low levels and necessary for activity; removal of the protein by binding to a high level of its cognate partner results in the same net effect, leading to inactivation of the protein and the associated signal transduction pathway. Recently, work done by Nicolaides et al. (Nicolaides N. C. et al. (1998) Mol. Cell. Biol. 18:1635-1641; U.S. Pat. No. 6,146,894 to Nicolaides et al.) has demonstrated the utility of introducing dominant negative inhibitory mismatch repair mutants into mammalian cells to confer global DNA hypermutability. The ability to manipulate the MMR process, and therefore, increase the mutability of the target host genome at will, in this example a mammalian cell, allows for the generation of innovative cell subtypes or variants of the original wild type cells. These variants can be placed under a specified, desired selective process, the result of which is a novel organism that expresses an altered biological molecule(s) and has a new trait. The concept of creating and introducing dominant negative alleles of a gene, including the MMR alleles, in bacterial cells has been documented to result in genetically altered prokaryotic mismatch repair genes (Aronshtam A. and M. G. Marinus (1996) Nucl. Acids Res. 24:2498-2504; Wu T. H. and M. G. Marinus (1994) J. Bacteriol. 176:5393-400; Brosh R. M. Jr. and S. W. Matson (1995) J. Bacteriol. 177:5612-5621).

Furthermore, altered MMR activity has been demonstrated when MMR genes from different species including yeast, mammalian cells, and plants are over-expressed (Fishel, R. et al. (1993) Cell 7:1027-1038; Studamire B. et al. (1998) Mol. Cell. Biol. 18:75907601; Alani E. et al. (1997) Mol. Cell. Biol. 17:2436-2447; Lipkin S. M. et al. (2000) Nat. Genet. 24:27-35).

Recently Guarne et al. (2001) EMBO J. 20(19):5521-5531 described the ATPase function of the MutLα, a heterodimer of MLH1 and PMS2. Guarne et al. studied the three dimensional structure of PMS2 and determined the portions of the molecule that participate in ATP binding and hydrolysis. Guarne et al. postulate that dimerization and ATPase activity are probably required for MMR function. Guarne et al., however, do not teach or suggest how their findings relate to dominant negative phenotypes of mismatch repair.

There is a continuing need in the art for methods of genetically manipulating cells to increase their performance characteristics and abilities. To this end, there is a need in the art to understand, develop and design MMR genes that confer a dominant negative effect for use in generating hypermutable cells.

### SUMMARY OF THE INVENTION

The invention provides methods of making a cell hypermutable comprising introducing into the cell a PMS2 homolog comprising a nucleotide sequence encoding a polypeptide comprising the amino acid sequence of SEQ ID NO:23, thereby making the cell hypermutable, wherein the PMS2 homolog is other than PMSR2 and PMSR3.

The invention also provides methods of making a mutation in a gene of interest comprising introducing into a cell containing a gene of interest a PMS2 homolog comprising a nucleotide sequence encoding a polypeptide wherein the polypeptide comprises the amino acid sequence of SEQ ID NO:23, thereby making said cell hypermutable, wherein the PMS2 homolog is other than PMSR2 and PMSR3, and selecting a mutant cell comprising a mutation in said gene of interest.

The invention also provides methods of making dominant negative MMR genes for introduction into cells to create hypermutable cells. The dominant negative MMR genes encode proteins comprising the amino acid sequence of SEQ ID NO:23 and share at least about 90% homology with PMS2-134 (SEQ ID NO:13).

The invention also provides methods of generating libraries of mutated genes. In embodiments of the methods of the invention, a dominant negative allele of a PMS2 homolog is introduced into a cell whereby the cell becomes hypermutable. The cells accumulate mutations in genes and a population of cells may therefore comprise a library of mutated genes as compared to wild-type cells with a stable genome.

In some embodiments of the methods of the invention, the polypeptides comprise the amino acid sequence of SEQ ID NO:24. In some embodiments, the polypeptides have a conserved ATPase domain. In some embodiments of the method of the invention the PMS2 homolog is a PMSR6. In certain embodiments of the method of the invention, the PMSR6 polypeptide comprises the amino acid sequence of SEQ ID NO:22 and is encoded by the polynucleotide sequence of SEQ ID NO:21.

In some embodiments of the methods of the invention, the PMS2 homolog further comprises a truncation which results in an inability to dimerize with MLH1. This may be a truncation from the E′ α-helix to the C-terminus, the E α-helix to the C-terminus, the F α-helix to the C-terminus, the G α-helix to the C-terminus, the H′ α-helix to the C-terminus, the H α-helix to the C-terminus, or the I α-helix to the C-terminus, for example, as described by Guarne et al. (2001) EMBO J. 20(19):5521-5531 and shown in FIG. 2.

The methods of the invention may be used for eukaryotic cells, particularly cells from protozoa, yeast, insects, vertebrates, and mammals, particularly humans. The methods of the invention may also be used for prokaryotic cells, such as bacterial cells, and may be used for plant cells.

The methods of the invention may also include treating the cells with a chemical mutagen or radiation to increase the rate of mutation over that observed by disrupting mismatch repair alone.

The hypermutable cells of the invention may be screened to detect a mutation in a gene of interest that confers a desirable phenotype. The cells may be screened by examining the nucleic acid, protein or the phenotype of the cells.

In some embodiments of the methods of the invention, genetic stability may be restored to the hypermutable cells, thereby maintaining cells comprising mutations in the gene of interest which may be further faithfully propagated.

The invention also provides methods of assaying cells to detect neoplasia comprising contacting said sample with a nucleotide sequence encoding the amino acid sequence of SEQ ID NO:23 to detect expression of a polynucleotide encoding a PMS2 homolog comprising the amino acid sequence of SEQ ID NO:23, wherein expression of said PMS2 homolog is associated with neoplasia. The detecting of the PMS2 homolog may be accomplished by any means known in the art, including but not limited to Northern blot analysis and RT-PCR.

The invention also provides methods of assaying cells to detect neoplasia comprising contacting said sample with an antibody directed against a PMS2 homolog or peptide fragments thereof; and detecting the presence of an antibody-complex formed with the PMS2 homolog or peptide fragment thereof, thereby detecting the presence of said PMS2 homolog in said sample, wherein the presence of said PMS2 homolog is associated with neoplasia. Methods of detection of PMS2 homologs may be by any means known in the art, including but not limited to radioimmunoassays, western blots, immunofluorescence assays, enzyme-linked immunosorbent assays (ELISA), and chemiluminescence assays.

The invention also provides methods of treating a patient with cancer comprising identifying a patient with a PMS2 homolog-associated neoplasm, administering to said patient an inhibitor of expression of said PMS2 homolog wherein said inhibitor suppresses expression of said PMS2 homolog in said PMS2 homolog associated neoplasm. Such neoplasms include, for example, lymphomas. Inhibitors of PMS2 homolog expression include antisense nucleotides, ribozymes, antibody fragments and ATPase analogs that specifically bind the PMS2 homolog.

### BRIEF DESCRIPTION OF THE FIGURES

FIG. 1 shows the polypeptide sequences of PMSR2, PMSR3 and PMSR6 showing consensus sequence regions with underlining. FIG. 1B shows an alignment of the consensus sequence region of PMS2 with a DNA gyrase-like ATPase motif.

FIG. 2A and B show the structure of the N-terminal fragment of PMS2 (orthagonal views) and FIG. 2C shows a sequence alignment of hPMS2, hMLH1 and MutL N-terminal fragments and structural features corresponding to FIGS. 2A and B (from Guarne et al. (2001) EMBO J. 20(19):5521-5531, FIG. 2A-C).

FIG. 3 shows RT-PCR analysis of PMSR genes in lymphoma cell lines. Thirty cycles of RT-PCR amplification was performed on lymphoma cell lines with (lanes 3-5) or without (lane 2) microsatellite instability (MI). As demonstrated above, each line with MI expressed either the PMSR2 or the PMSR3 gene, while no expression was observed in cell lines lacking MI (lane 2). hPMS2 and β-actin message was used as internal controls to measure for RNA loading. Lane 1 was a mock reaction to measure for potential artifact or contamination. Additional PCR amplifications were performed using 45 cycles of amplification which resulted in more robust products in positive lanes, as observed with 30 cycles, while no PMSR signal was detected in negative samples such as those presented in lanes 1 and 2.

FIG. 4 shows Western blot analysis of human lymphoma cell lines with (LMM-1) (lane 2) or without (LNM-a) (lane 1) microsatellite instability (MI). The arrows indicate proteins with the expected molecular weight of the hPMS2 and hPMSR2 polypeptides. A correlation of PMSR expression is observed in lymphoma cell lines exhibiting MI.

FIG. 5 shows β-galactosidase activity in 293 cells expressing PMS2 and PMSR homologs plus the MMR-sensitive pCAR-OF reporter. Cells in which MMR activity is decreased results in MI leading to insertion-deletion mutations within the β-galactosidase gene, a subset of which will restore the open reading frame (ORF) and produce functional enzyme. Cells are grow for 17 days and then harvested for protein lysates to measure β-galactosidase activity generated by each cell line. Cells in which a high rate of mutagenesis has occurred will produce β-galactosidase activity, while cells in which MMR activity is functional will retain background levels of enzymatic activity. Each cell line was tested in two independent experiments (experiment 1 and experiment 2). Extracts were incubated with a calorimetric galactose substrate for 1 hour. Enzyme activity as a function of substrate conversion was measured by optical density at 576 nm as described (Nicolaides, N. C. et al. (1998) Mol. Cell. Biol. 18:1635-1641). As shown above, cells expressing PMSR2 and PMSR3 had a high degree of MI leading to an increase in β-galactosidase activity. MI was monitored at the gene level to confirm that genetic alterations occurred within the polynucleotide repeat disrupting the β-galactosidase ORF.

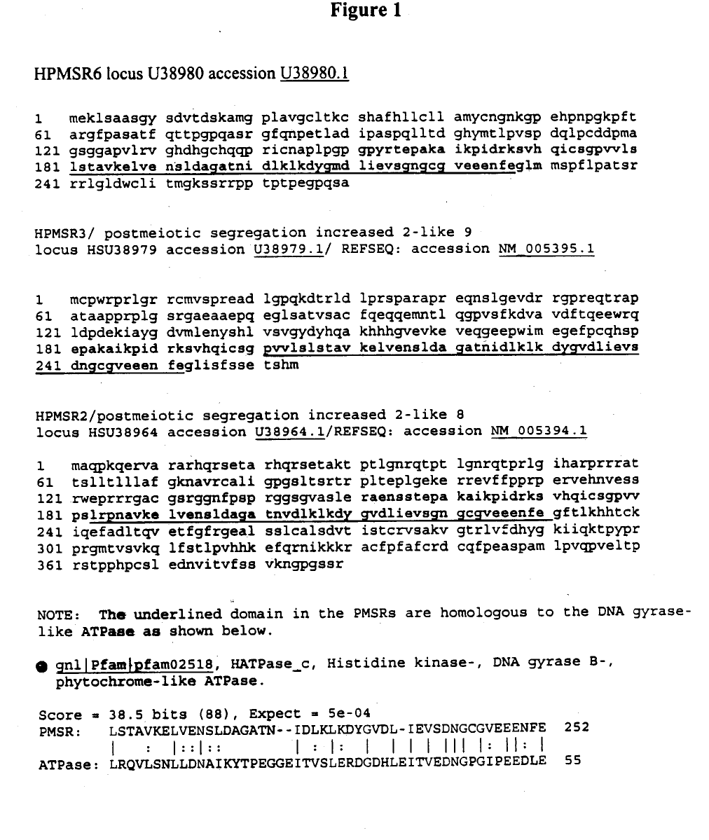

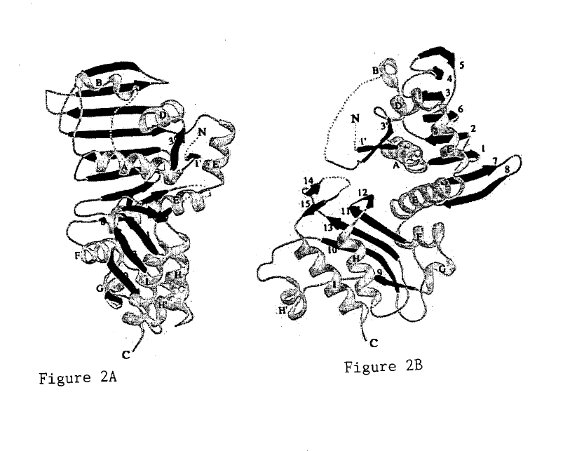

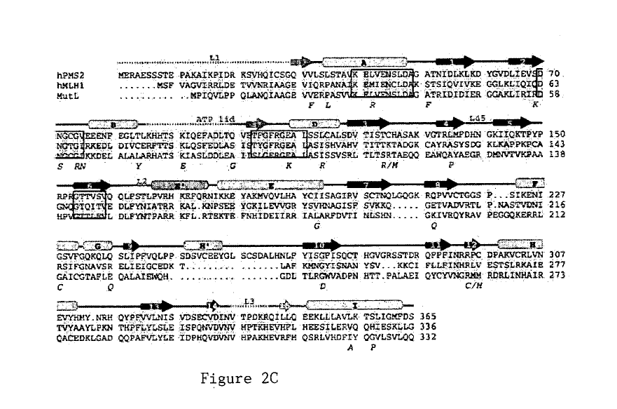

### DETAILED DESCRIPTION OF THE INVENTION

The present invention provides methods of making cells hypermutable using derivatives of mismatch repair genes bearing a consensus sequence for an ATPase. The consensus sequence is present in a number of PMS2 homologs that confers a dominant negative phenotype of mismatch repair when transfected into host cells.

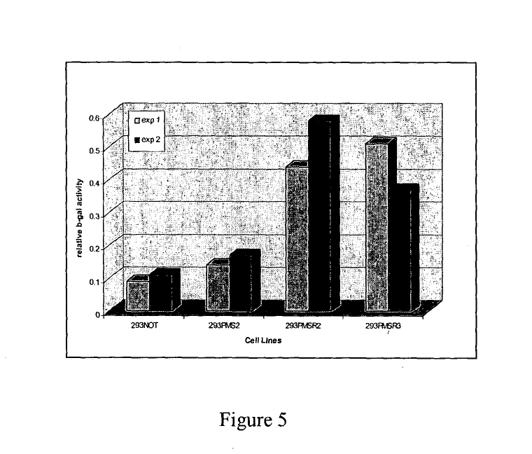

PMS2 homologs, such as PMSR2 and PMSR3 encode homologs of the mutL mismatch repair family of proteins. Both PMSR2 and PMSR3 proteins, for example, are highly homologous to the N-terminus of the human PMS2 gene and its encoded polypeptide. Functional studies have shown that when the PMSR2 or PMSR3 cDNAs are expressed in MMR proficient mammalian cells either of these homologs are capable of inactivating MMR in a dominant negative fashion resulting in genetic instability (see FIG. 5).

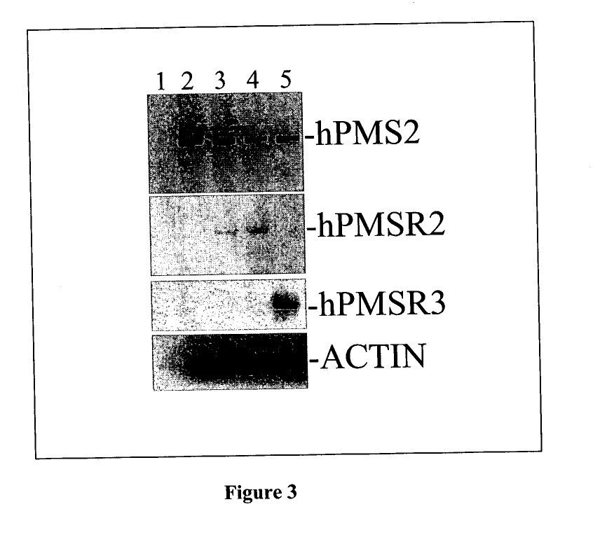

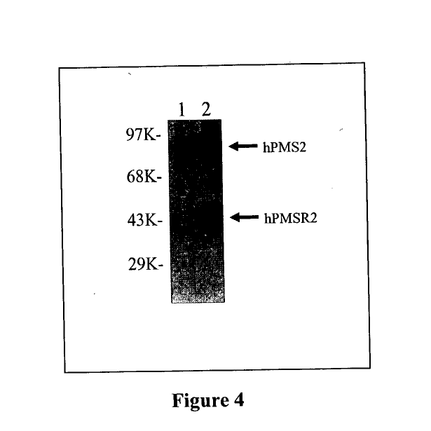

Preliminary gene expression studies have found that the PMSR2 or PMSR3 genes are not expressed in non-neoplastic tissues and are only detected in a subset of human lymphoma cell lines, of Burkitt's lymphoma origin, that exhibit microsatellite instability, a hallmark of MMR deficiency (see FIGS. 3 and 4).

The present invention is directed to use of PMS2 homologs which comprise the conserved domain of PMS134, PMSR2 and PMSR3 and share conserved portions of ATPase domains for use in generating hypermutable cells by introducing into cells polynucleotide sequences encoding PMS2 homologs which function to decrease MMR activity in a dominant negative fashion.

It has been discovered that proteins comprising a consensus sequence and homology to the N-terminal domain of PMS2, including structural features of ATPase domains function as dominant negative mismatch repair inhibitors. In a specific embodiment, PMSR6 expression confers a dominant negative phenotype of MMR deficiency in cells.

It has further been discovered that proteins comprising the consensus sequence of SEQ ID NO:23 or SEQ ID NO:24 and comprising a portion having at least about 90% homology with PMS2-134 can confer a dominant negative phenotype and a reduction in MMR activity when introduced into cells. In some embodiments the PMS2 homologs comprise ATPase domains. The PMS2 homologs may further comprise domains that are other than MMR proteins, such as chimeric or fusion proteins comprising a domain that is homologous to PMS2-134 and a portion that is heterologous.

As used herein, the term “PMS2 homolog” refers to a polypeptide sequence having the consensus sequence of AVKE LVENSLDAGA TN (SEQ ID NO:23). In some embodiments, the PMS2 homologs comprise the polypeptide sequence of LRPNAVKE LVENSLDAGA TNVDLKLKDY GVDLIEVSGN GCGVEEENFE (SEQ ID NO:24). The PMS2 homologs comprise this structural feature and, while not wishing to be bound by any particular theory of operation, it is believed that this structural feature correlates with ATPase activity due to the high homology with known ATPases. The knowledge of this structural feature and correlated function and the representative number of examples provided herein, will allow one of ordinary skill in the art to readily identify which proteins may be used in the methods of the invention.

As used herein a “nucleic acid sequence encoding a PMS2 homolog” refers to a nucleotide sequence encoding a polypeptide having the ATPase consensus sequence motifs and that, when expressed in a cell decreases the activity of mismatch repair in the cell. The nucleic acid sequences encoding the PMS2 homologs, when introduced and expressed in the cells, increase the rate of spontaneous mutations by reducing the effectiveness of endogenous mismatch repair mediated DNA repair activity, thereby rendering the cell highly susceptible to genetic alterations, (i.e., render the cells hypermutable). Hypermutable cells can then be utilized to screen for mutations in a gene or a set of genes in variant siblings that exhibit an output trait(s) not found in the wild-type cells. The PMS2 homologs may be an altered mismatch repair genes, or may be a mismatch repair gene that when overexpressed in the cell results in an impaired mismatch repair activity.

The nucleic acid sequences encoding the PMS2 homologs are introduced into the cells and expressed. The cell's mismatch repair activity is decreased and the cell becomes hypermutable. In some embodiments, the cells may be further incubated with a chemical mutagen to further enhance the rate of mutation.

While it has been documented that MMR deficiency can lead to as much as a 1000-fold increase in the endogenous DNA mutation rate of a host, there is no assurance that MMR deficiency alone will be sufficient to alter every gene within the DNA of the host bacterium to create altered biochemicals with new activity(s). Therefore, the use of chemical mutagens and their respective analogues such as ethidium bromide, EMS, MNNG, MNU, Tamoxifen, 8-Hydroxyguanine, as well as others such as those taught in: Khromov-Borisov, N. N., et al. (1999) Mutat. Res. 430:55-74); Ohe, T. et al. (1999) Mutat. Res. 429:189-199); Hour, T. C. et al. (1999) Food Chem. Toxicol. 37:569-579); Hrelia, P. et al. (1999) Chem. Biol. Interact. 118:99111); Garganta, F. et al. (1999) Environ. Mol. Mutagen. 33:75-85); Ukawa-Ishikawa S. et al. (1998) Mutat. Res. 412:99-107; www.ehs.utah.edu/ohh/mutagens, etc. can be used to further enhance the spectrum of mutations and increase the likelihood of obtaining alterations in one or more genes that can in turn generate host cells with a desired new output trait(s). Mismatch repair deficiency leads to hosts with an increased resistance to toxicity by chemicals with DNA damaging activity. This feature allows for the creation of additional genetically diverse hosts when mismatch defective cells are exposed to such agents, which would be otherwise impossible due to the toxic effects of such chemical mutagens [Colella, G. et al. (1999) Br. J. Cancer 80:338-343); Moreland, N. J. et al. (1999) Cancer Res. 59:2102-2106); Humbert, O. et al. (1999) Carcinogenesis 20:205-214); Glaab, W. E. et al. (1998) Mutat. Res. 398:197-207].

The cells that may be transfected with the PMS2 homologs include any prokaryotic or eukaryotic cell. The prokaryotic cells may be bacterial cells of a wide array of genera.

In other embodiments, the cells are eukaryotic cells, such as, but not limited to insect cells, protozoans, yeast, fungi, vertebrate cells (such as, for example, fish, avian, reptilian and amphibian cells), mammalian cells (including, for example, human, non-human primate, rodent, caprine, equine, bovine, and ovine cells).

In other embodiments, plant cells may be transfected with a PMS2 homolog to render the plant cells hypermutable.

Once cells are rendered hypermutable, the genome of the cells will begin to accumulate mutations, including mutations in genes of interest. The mutations in the genes of interest may confer upon these genes desirable new phenotypes that can be selected. As a non-limiting example, mutations in protein-encoding genes may render the proteins expressed at higher levels. As another non-limiting example, proteins such as antibodies and enzymes may have altered binding characteristics, such as higher affinities for their antigen or substrate, respectively. Such altered phenotypes may be screened and the cells containing the genes and displaying the altered phenotypes may be selected for further cultivation.

The genome of the cells containing the genes of interest with new phenotype may be rendered genetically stable by counteracting the effects of the transfected PMS2 homologs. Those of skill in the art may “cure” the cells of plasmids that contain the PMS2 homologs or disrupt the PMS2 homolog within the cell such that the PMS2 homolog is no longer expressed. Plasmids that are maintained in cells only under drug pressure may be used to cultivate the cells with PMS2 homologs. When the drug pressure is removed the cells tend to lose the plasmids. In other embodiments, inducible expression vectors may be used to express the PMS2 homologs. Thereafter, the inducer molecule may be withdrawn to allow the genome to stabilize.

As used herein, the term “mismatch repair,” also called “mismatch proofreading,” refers to an evolutionarily highly conserved process that is carried out by protein complexes described in cells as disparate as prokaryotic cells such as bacteria to more complex mammalian cells (Modrich, P. (1994) Science 266:1959-1960; Parsons, R. et al. (1995) Science 268:738-740; Perucho, M. (1996) Biol Chem. 377: 675-684). A mismatch repair gene is a gene that encodes one of the proteins of such a mismatch repair complex. Although not wanting to be bound by any particular theory of mechanism of action, a mismatch repair complex is believed to detect distortions of the DNA helix resulting from non-complementary pairing of nucleotide bases. The non-complementary base on the newer DNA strand is excised, and the excised base is replaced with the appropriate base that is complementary to the older DNA strand. In this way, cells eliminate many mutations that occur as a result of mistakes in DNA replication, resulting in genetic stability of the sibling cells derived from the parental cell.

Some wild type alleles as well as dominant negative alleles cause a mismatch repair defective phenotype even in the presence of a wild-type allele in the same cell. An example of a dominant negative allele of a mismatch repair gene is the human gene hPMS2-134, which carries a truncation mutation at codon 134 (Parsons, R. et al. (1995) Science 268:738-740; Nicolaides N. C. et al (1998) Mol. Cell. Biol. 18:1635-1641). The mutation causes the product of this gene to abnormally terminate at the position of the 134^{th} amino acid, resulting in a shortened polypeptide containing the N-terminal 133 amino acids. Such a mutation causes an increase in the rate of mutations, which accumulate in cells after DNA replication. Expression of a dominant negative allele of a mismatch repair gene results in impairment of mismatch repair activity, even in the presence of the wild-type allele. Any PMS2 homolog, which produces such effect, can be used in this invention, whether it is wild-type or altered, whether it derives from mammalian, yeast, fungal, amphibian, insect, plant, bacteria or is designed as a chimera or fusion protein.

Yeast, for example, which may be the source of host MMR, may be mutated or not. The term “yeast” used in this application comprises any strain from the eukaryotic kingdom, including but not limited to Saccharomyces sp., Pichia sp., Schizosaccharomyces sp., Kluyveromyces sp., and other fungi (Gellissen, G. and Hollenberg, C. P. (1997) Gene 190(1):87-97). These organisms can be exposed to chemical mutagens or radiation, for example, and can be screened for defective mismatch repair. Genomic DNA, cDNA, mRNA, or protein from any cell encoding a mismatch repair protein can be analyzed for variations from the wild-type sequence. Dominant negative alleles of PMS2 homologs can also be created artificially, for example, by creating fusion proteins or chimeric proteins in which a portion of the protein comprises the consensus sequence of SEQ ID NO:23 or SEQ ID NO:24, has about 90% amino acid homology with PMS2-134, and another portion that is a heterologous amino acid sequence.

Various techniques of site-directed mutagenesis can be used. The suitability of such alleles, whether natural or artificial, for use in generating hypermutable yeast can be evaluated by testing the mismatch repair activity (using methods described in Nicolaides N. C. et al. (1998) Mol. Cell. Biol. 18:1635-1641) caused by the allele in the presence of one or more wild-type alleles to determine if it is a dominant negative allele.

A cell that over-expresses a wild type mismatch repair allele or a dominant negative allele of a mismatch repair gene will become hypermutable. This means that the spontaneous mutation rate of such cell is elevated compared to cells without such alleles. The degree of elevation of the spontaneous mutation rate can be at least 2-fold, 5-fold, 10-fold, 20-fold, 50-fold, 100-fold, 200-fold, 500-fold, or 1000-fold that of the normal cell as measured as a function of cell doubling/hour.

According to one aspect of the invention, a polynucleotide encoding the PMS2 homolog is introduced into a cell such as a mammalian cell, vertebrate cell, plant cell, or yeast, for example. The gene is a PMS2 homolog and is a dominant negative. The PMS2 homolog can be naturally occurring or made in the laboratory. The polynucleotide can be in the form of genomic DNA, cDNA, RNA, or a chemically synthesized polynucleotide or polypeptide. The molecule can be introduced into the cell by transformation, electroporation, mating, particle bombardment, or other method described in the literature.

Transformation is used herein as any process whereby a polynucleotide or polypeptide is introduced into a cell. The process of transformation can be carried out in a yeast culture using a suspension of cells.

In general, transformation will be carried out using a suspension of cells but other methods can also be employed as long as a sufficient fraction of the treated cells incorporate the polynucleotide or polypeptide so as to allow transfected cells to be grown and utilized. The protein product of the polynucleotide may be transiently or stably expressed in the cell. Techniques for transformation are well known to those skilled in the art. Available techniques to introduce a polynucleotide or polypeptide into a cell include but are not limited to electroporation, viral transduction, cell fusion, the use of spheroplasts or chemically competent cells (e.g., calcium chloride), and packaging of the polynucleotide together with lipid for fusion with the cells of interest. Once a cell has been transformed with the mismatch repair gene or protein, the cell can be propagated and manipulated in either liquid culture or on a solid agar matrix, such as a petri dish. If the transfected cell is stable, the gene will be expressed at a consistent level for many cell generations, and a stable, hypermutable yeast strain results.

An isolated yeast cell can be obtained from a yeast culture by chemically selecting strains using antibiotic selection of an expression vector. If the yeast cell is derived from a single cell, it is defined as a clone. Techniques for single-cell cloning of microorganisms such as yeast are well known in the art.

A polynucleotide encoding a PMS2 homolog can be introduced into the genome of yeast or propagated on an extra-chromosomal plasmid, such as the 2-micron plasmid. Selection of clones harboring a mismatch repair gene expression vector can be accomplished by plating cells on synthetic complete medium lacking the appropriate amino acid or other essential nutrient as described (Schneider, J. C. and L. Guarente (1991) Methods in Enzymology 194:373). The yeast can be any species for which suitable techniques are available to produce transgenic microorganisms, such as but not limited to genera including Saccharomyces, Schizosaccharomyces, Pichia, Hansenula, Kluyveromyces and others.

Any method for making transgenic yeast known in the art can be used. According to one process of producing a transgenic microorganism, the polynucleotide is introduced into the yeast by one of the methods well known to those in the art. Next, the yeast culture is grown under conditions that select for cells in which the polynucleotide encoding the mismatch repair gene is either incorporated into the host genome as a stable entity or propagated on a self-replicating extra-chromosomal plasmid, and the protein encoded by the polynucleotide fragment transcribed and subsequently translated into a functional protein within the cell. Once transgenic yeast is engineered to harbor the expression construct, it is then propagated to generate and sustain a culture of transgenic yeast indefinitely.

Once a stable, transgenic cell has been engineered to express a PMS2 homolog, the cell can be cultivated to create novel mutations in one or more target gene(s) of interest harbored within the same cell. A gene of interest can be any gene naturally possessed by the cell or one introduced into the cell host by standard recombinant DNA techniques. The target gene(s) may be known prior to the selection or unknown. One advantage of employing transgenic yeast cells to induce mutations in resident or extra-chromosomal genes within the yeast is that it is unnecessary to expose the cells to mutagenic insult, whether it is chemical or radiation, to produce a series of random gene alterations in the target gene(s). This is due to the highly efficient nature and the spectrum of naturally occurring mutations that result as a consequence of the altered mismatch repair process. However, it is possible to increase the spectrum and frequency of mutations by the concomitant use of either chemical and/or radiation together with MMR defective cells. The net effect of the combination treatment is an increase in mutation rate in the genetically altered yeast that are useful for producing new output traits. The rate of the combination treatment is higher than the rate using only the MMR-defective cells or only the mutagen with wild-type MMR cells. The same strategy is useful for other types of cells including vertebrate and mammalian cells.

MMR-defective cells of the invention can be used in genetic screens for the direct selection of variant sub-clones that exhibit new output traits with commercially desirable applications. This permits one to bypass the tedious and time-consuming steps of gene identification, isolation and characterization.

Mutations can be detected by analyzing the internally and/or externally mutagenized cells for alterations in its genotype and/or phenotype. Genes that produce altered phenotypes in MMR-defective microbial cells can be discerned by any of a variety of molecular techniques well known to those in the art. For example, the cell genome can be isolated and a library of restriction fragments of the yeast genome can be cloned into a plasmid vector. The library can be introduced into a “normal” cell and the cells exhibiting the novel phenotype screened. A plasmid can be isolated from those normal cells that exhibit the novel phenotype and the gene(s) characterized by DNA sequence analysis.

Alternatively, differential messenger RNA screen can be employed utilizing driver and tester RNA (derived from wild type and novel mutant, respectively) followed by cloning the differential transcripts and characterizing them by standard molecular biology methods well known to those skilled in the art. Furthermore, if the mutant sought is encoded by an extra-chromosomal plasmid, then following co expression of the dominant negative MMR gene and the gene of interest, and following phenotypic election, the plasmid can be isolated from mutant clones and analyzed by DNA sequence analysis using methods well known to those in the art.

Phenotypic screening for output traits in MMR-defective mutants can be by biochemical activity and/or a readily observable phenotype of the altered gene product. A mutant phenotype can also be detected by identifying alterations in electrophoretic mobility, DNA binding in the case of transcription factors, spectroscopic properties such as IR, CD, X-ray crystallography or high field NMR analysis, or other physical or structural characteristics of a protein encoded by a mutant gene. It is also possible to screen for altered novel function of a protein in situ, in isolated form, or in model systems. One can screen for alteration of any property of the yeast associated with the function of the gene of interest, whether the gene is known prior to the selection or unknown.

The screening and selection methods discussed are meant to illustrate the potential means of obtaining novel mutants with commercially valuable output traits, but they are not meant to limit the many possible ways in which screening and selection can be carried out by those of skill in the art.

Plasmid expression vectors that harbor a PMS2 homolog insert can be used in combination with a number of commercially available regulatory sequences to control both the temporal and quantitative biochemical expression level of the dominant negative MMR protein. The regulatory sequences can be comprised of a promoter, enhancer or promoter/enhancer combination and can be inserted either upstream or downstream of the MMR gene to control the expression level. The regulatory sequences can be any of those well known to those in the art for extra-chromosomal expression vectors or on constructs that are integrated into the genome via homologous recombination. These types of regulatory systems have been disclosed in scientific publications and are familiar to those skilled in the art.

Once a cell with a novel, desired output trait of interest is created, the activity of the aberrant MMR activity is desirably attenuated or eliminated by any means known in the art. These include but are not limited to removing an inducer from the culture medium that is responsible for promoter activation, curing a plasmid from a transformed yeast cell, and addition of chemicals, such as 5-fluoro orotic acid to “loop-out” the gene of interest.

In the case of an inducibly controlled dominant negative PMS2 homolog, expression of the PMS2 homolog will be turned on (induced) to generate a population of hypermutable cells with new output traits. Expression of the dominant negative MMR allele can be rapidly turned off to reconstitute a genetically stable strain that displays a new output trait of commercial interest. The resulting cell is now useful as a stable cell line that can be applied to various commercial applications, depending upon the selection process placed upon it.

In cases where genetically deficient mismatch repair cell are used to derive new output traits, transgenic constructs can be used that express wild type mismatch repair genes sufficient to complement the genetic defect and therefore restore mismatch repair activity of the host after trait selection [Grzesiuk, E. et al. (1998) Mutagenesis 13:127-132); Bridges, B. A. et al. (1997) EMBO J. 16:3349-3356); LeClerc, J. E. (1996) Science 15:1208-1211); Jaworski, A. et al. (1995) Proc. Natl. Acad. Sci USA 92:11019-11023]. The resulting cell is genetically stable and can be employed for various commercial applications.

The use of over-expression of foreign (exogenous, transgenic) mismatch repair genes from human and yeast such as MSH2, MLH1, MLH3, etc. have been previously demonstrated to produce a dominant negative mutator phenotype in yeast hosts (Shcherbakova, P. V. et al. (2001) Mol. Cell. Biol. 21(3):940-951; Studamire, B. et al. (1998) Mol. Cell. Biol. 18:7590-7601; Alani E. et al. (1997) Mol. Cell. Biol. 17:2436-2447; Lipkin, S. M. et al. (2000) Nat. Genet. 24:27-35). In addition, the use of yeast strains expressing prokaryotic dominant negative MMR genes as well as hosts that have genomic defects in endogenous MMR proteins have also been previously shown to result in a dominant negative mutator phenotype (Evans, E. et al. (2000) Mol. Cell. 5(5):7897-7899; Aronshtam A. and M. G. Marinus (1996) Nucl. Acids Res. 24:2498-2504; Wu, T. H. and M. G. Marinus (1994) J. Bacteriol. 176:5393-5400; Brosh R. M. Jr., and S. W. Matson (1995) J. Bacteriol. 177:5612-5621). However, the findings disclosed here teach the use of PMS2 homologs, including the human PMSR2 gene (Nicolaides, N. C. et al. (1995) Genomics 30:195-206), the related PMS2-134 truncated MMR gene (Nicolaides N. C. et al. (1995) Genomics 29:329-334), the plant mismatch repair genes (U.S. patent application Ser. No. 09/749,601) and those genes that are homologous to the 134 N-terminal amino acids of the PMS2 gene to create hypermutable yeast.

The ability to create hypermutable organisms using PMS2 homologs can be used to generate innovative yeast strains that display new output features useful for a variety of applications, including but not limited to the manufacturing industry, for the generation of new biochemicals, for detoxifying noxious chemicals, either by-products of manufacturing processes or those used as catalysts, as well as helping in remediation of toxins present in the environment, including but not limited to polychlorobenzenes (PCBs), heavy metals and other environmental hazards. Novel cell lines can be selected for enhanced activity to either produce increased quantity or quality of a protein or non-protein therapeutic molecule by means of biotransformation. Biotransformation is the enzymatic conversion of one chemical intermediate to the next intermediate or product in a pathway or scheme by a microbe or an extract derived from the microbe. There are many examples of biotransformation in use for the commercial manufacturing of important biological and chemical products, including penicillin G, erythromycin, and clavulanic acid. Organisms that are efficient at conversion of “raw” materials to advanced intermediates and/or final products also can perform biotransformation (Berry, A. (1996) Trends Biotechnol. 14(7):250-256). The ability to control DNA hypermutability in host cells using a PMS2 homolog allows for the generation of variant subtypes that can be selected for new phenotypes of commercial interest, including but not limited to organisms that are toxin-resistant, have the capacity to degrade a toxin in situ or the ability to convert a molecule from an intermediate to either an advanced intermediate or a final product.

Other applications using PMS2 homologs to produce genetic alteration of host cells for new output traits include but are not limited to recombinant production strains that produce higher quantities of a recombinant polypeptide as well as the use of altered endogenous genes that can transform chemical or catalyze manufacturing downstream processes. A regulatable PMS2 homolog can be used to produce a cell with a commercially beneficial output trait. Using this process, cells expressing a PMS2 homolog can be directly selected for the phenotype of interest. Once a selected cell with a specified output trait is isolated, the hypermutable activity can be turned-off by several methods well known to those skilled in the art. For example, if the PMS2 homolog is expressed by an inducible promoter system, the inducer can be removed or depleted. Such systems include but are not limited to promoters such as: lactose inducibleGALi-GAL10 promoter (Johnston, M. and R. W. Davis (1984) Mol. Cell Biol. 4:1440); the phosphate inducible PH05 promoter (Miyanohara, A. et al. (1983) Proc. Natl. Acad. Sci. USA 80:1-5); the alcohol dehydrogenase I (ADH) and 3-phosphoglycerate kinase (PGK) promoters, that are considered to be constitutive but can be repressed/de-repressed when yeast cells are grown in non-fermentable carbon sources such as but not limited to lactate (Ammerer, G. (1991) Methods in Enzymology 194:192; Mellor, J. et al. (1982) Gene 24:563); Hahn S. and L. Guarente (1988) Science 240:317); Alcohol oxidase (AOX) in Pichia pastoris (Tschopp, J. F. et al (1987) Nucl. Acids Res. 15(9):3859-76; and the thiamine repressible expression promoter nmtl in Schizosaccharomyces pombe (Moreno, M. B. et al. (2000) Yeast 16(9):861-872). Yeast cells can be transformed by any means known to those skilled in the art, including chemical transformation with LiCI (Mount, R. C. et al. (1996) Methods Mol. Biol. 53:139-145) and electroporation (Thompson, J. R. et al. (1998) Yeast 14(6):565-571). Yeast cells that have been transformed with DNA can be selected for growth by a variety of methods, including but not restricted to selectable markers (URA3; Rose, M. et al. (1984) Gene 29:113; LEU2; Andreadis, A. et al. (1984) J. Biol. Chem. 259:8059; ARG4; Tschumper G. and J. Carbon (1980) Gene 10:157; and HIS3; Struhl, K. et al. (1979) Proc. Natl. Acad. Sci. USA 76:1035) and drugs that inhibit growth of yeast cells (tunicamycin, TUN; Hahn, S. et al. (1988) Mol. Cell Biol. 8:655). Recombinant DNA can be introduced into yeast as described above and the yeast vectors can be harbored within the yeast cell either extra-chromosomally or integrated into a specific locus. Extra-chromosomal based yeast expression vectors can be either high copy based (such as the 2-pm vector Yep13; Rose, A. B. and J. R. Broach (1991) Methods in Enzymology 185:234), low copy centromeric vectors that contain autonomously replicating sequences (ARS) such as YRp7 (Fitzgerald-Hayes, M. et al. (1982) Cell 29:235) and well as integration vectors that permit the gene of interest to be introduced into specified locus within the host genome and propagated in a stable manner (Rothstein, R. J. (1991) Methods in Enzymology 101:202). Ectopic expression of MMR genes in yeast can be attenuated or completely eliminated at will by a variety of methods, including but not limited to removal from the medium of the specific chemical inducer (e.g., deplete galactose that drives expression of the GAL10 promoter in Saccharomyces cerevisiae or methanol that drives expression of the AOX1 promoter in Pichia pastoris), extrachromosomally replicating plasmids can be “cured” of expression plasmid by growth of cells under non-selective conditions (e.g., YEp13 harboring cells can be propagated in the presence of leucine,) and cells that have genes inserted into the genome can be grown with chemicals that force the inserted locus to “loop-out” (e.g., integrants that have URA3 can be selected for loss of the inserted gene by growth of integrants on 5-fluoroorotic acid (Boeke, J. D. et al. (1984) Mol. Gen. Genet. 197:345-346). Whether by withdrawal of inducer or treatment of yeast cells with chemicals, removal of MMR expression results in the reestablishment of a genetically stable yeast cell-line. Thereafter, the lack of mutant MMR allows the endogenous, wild type MMR activity in the host cell to function normally to repair DNA. The newly generated mutant yeast strains that exhibit novel, selected output traits are suitable for a wide range of commercial processes or for gene/protein discovery to identify new biomolecules that are involved in generating a particular output trait. Of course, yeast is only one example of cell types that may be used and similar strategies using known promoters and inducers may be employed for use in other types of cells including vertebrate, insect, and mammalian cells, for example.

Moreover, mismatch repair is responsible for repairing chemically-induced DNA adducts, therefore blocking this process could theoretically increase the number, types, mutation rate and genomic alterations of a yeast [Rasmussen, L. J. et al. (1996) Carcinogenesis 17:2085-2088); Sledziewska Gojska, E. et al. (1997) Mutat. Res. 383:31-37); and Janion, C. et al. (1989) Mutat. Res. 210:15-22)]. In addition to the chemicals listed above, other types of DNA mutagens include ionizing radiation and UV irradiation, which is known to cause DNA mutagenesis in yeast, can also be used to potentially enhance this process (Lee C. C. et al (1994) Mutagenesis 9:401-405; Vidal A. et al. (1995) Carcinogenesis 16:817-821). These agents, which are extremely toxic to host cells and therefore result in a decrease in the actual pool size of altered yeast cells are more tolerated in MMR defective hosts and in turn permit an enriched spectrum and degree of genomic mutagenesis.

The general methods of the invention therefore also provide a method of generating libraries of mutated genes in which the cells made hypermutable from the introduction of the PMS2 homologs accumulate mutations and may be used subsequently to produce cDNA and genomic libraries comprising mutated genes (as compared to the wild-type parental host cells). Methods of preparing cDNA and genomic libraries are well known in the art and techniques may be found, for example in Sambrook et al. MOLECULAR CLONING: A LABORATORY MANUAL, Third Edition, 2001.

The invention also provides methods of assaying cells to detect neoplasia comprising contacting said sample with a nucleotide sequence encoding the amino acid sequence of SEQ ID NO:23 to detect expression of a polynucleotide encoding a PMS2 homolog comprising the amino acid sequence of SEQ ID NO:23, wherein expression of said PMS2 homolog is associated with neoplasia.

The PMS2 homolog is identified as having the consensus sequence of SEQ ID NO:23 or SEQ ID NO:24 and may be detected by nucleic acids comprising a sequence that encodes SEQ ID NO:23 or SEQ ID NO:24. One of ordinary skill in the art may design reverse transcriptase-polymerase chain reaction assays (RT-PCR assays) to detect the expression of the PMS2 homologs in the cells suspected of being neoplastic. Northern blots may also be used to detect PMS2 homolog expression using standard protocols such as those found in, for example, Sambrook et al. MOLECULAR CLONING: A LABORATORY MANUAL, Third Edition, 2001.

The invention also provides methods of assaying cells to detect neoplasia comprising contacting said sample with an antibody directed against a PMS2 homolog or peptide fragments thereof; and detecting the presence of an antibody-complex formed with the PMS2 homolog or peptide fragment thereof, thereby detecting the presence of said PMS2 homolog in said sample, wherein the presence of said PMS2 homolog is associated with neoplasia. Methods of detection of PMS2 homologs may be by any means known in the art, including but not limited to radioimmunoassays, western blots, immunofluorescence assays, enzyme-linked, immunosorbent assays (ELISA), and chemiluminescence assays. The various protocols for these assays are well-known in the art.

The invention also provides methods of treating a patient with cancer comprising identifying a patient with a PMS2 homolog-associated neoplasm, administering to said patient an inhibitor of expression of said PMS2 homolog wherein said inhibitor suppresses expression of said PMS2 homolog in said PMS2 homolog associated neoplasm. Such neoplasms include, for example, lymphomas. Inhibitors of PMS2 homolog expression include antisense nucleotides, ribozymes, antibody fragments and ATPase analogs that specifically bind the PMS2 homolog.

The antisense molecules are polynucleotides that are complementary to a portion of the RNA encoding the PMS2 homolog and bind specifically to the RNA. The antisense molecules inhibit the translation of the PMS2 homolog RNA and thereby inhibit the effect of PMS2 expression. Antisense molecules may be directed to portions of the RNA that are involved in robosome binding or initiation of translation as well as to portions of the coding sequence. Generally antisense molecules are at least 15 nucleotides in length, but may be 20, 25, 30, 35, 40, 45, 50 or more nucleotides in length.

Ribozymes are a special catalytic class of antisense molecules that cleave substrate nucleotides. Design of ribozymes for PMS2 homologs may be performed using methods well-known in the art, as described, for example in Lyngstadaas S P. (2001) “Synthetic hammerhead ribozymes as tools in gene expression” Crit. Rev. Oral. Biol. Med. 12(6):469-78; Samarsky D, Ferbeyre G, Bertrand E. (2000) “Expressing active ribozymes in cells” Curr. Issues Mol. Biol. 2(3):87-93. The ribozyme or vector encoding a ribozyme are introduced into the cells expressing the PMS2 homolog and are activated such that the ribozyme binds to and cleaves the polynucleotide encoding the PMS2 homolog, thereby preventing expression of the PMS2 homolog.

The above disclosure generally describes the present invention. A more complete understanding can be obtained by reference to the following specific examples that will be provided herein for purposes of illustration only, and are not intended to limit the scope of the invention.

### EXAMPLES

### Example 1

### Evaluation The Association Of PMSR2 And PMSR3 RNA Expression In Tumors Of Lymphoid Tissue and Comparison With Microsatellite Instability Profile

A panel of lymphoma tissues and cell lines are analyzed for microsatellite instability (MI) by PCR mediated genotypic analysis and for PMSR2 and PMSR3 expression via RT-PCR analysis following methods previously used and described in publications by Dr. Nicolaides (Liu, B. et al. (1996) Nature Med. 2:169-174; Nicolaides, N. C. et al. (1996) Genomics 31:395-397). For RNA expression studies, RNAs are extracted from a panel of 83 lymphoma cell lines (obtained from ATCC and personal contacts) using the trizol method as described by the manufacturer (Gibco/BRL). 100 ngs of total RNA are reverse transcribed using SuperscriptII reverse transcriptase (RT) and random hexamers as primer in 20 μl reactions as recommended by the manufacturer (Gibco/BRL). Each sample is incubated in reaction buffer with (RT+) or without (RT−) RT, where the RT− samples serve as negative control. Reactions are incubated for 1 hour at 37° C. and diluted to a final volume of one hundred microliters. Routinely, 5 μls of each sample is used for PCR amplification in 25 μl reactions containing 67 mM Tris, pH 8.8, 16.6 mM (NH₄)₂SO₄, 6.7 mM MgCl₂, 10 mM 2-mercaptoethanol, 4% DMSO, 1.25 mM each of the four dNTPs, 175 ng of each cDNA specific primer and 1 U of Taq polymerase. Amplifications are carried out at 94° C. for 30 sec, 58° C. for 90 sec, 72° C. for 90 sec for 30 cycles. One half of the reaction is loaded onto 1% agarose gels in 1×Tris Acetate EDTA running buffer and detected by ethidium bromide staining. Below is a table (Table 1) with the gene specific primers and expected molecular weight PCR fragments. Samples are scored positive if an RT+ reaction contains a DNA fragment of the expected molecular weight while no signal is observed in RT− or water controls.

TABLE 1
Primers for specific amplification of PMSR cDNAs from cells and tissues.

| Gene | Forward primer | Reverse primer | Size (bp) |
|---|---|---|---|
| hPMS2 | 5′-ggacgagaagtataacttcgag-3′ | 5′-catctcgcttgtgttaagagc-3′ | 372 |
| | (SEQ ID NO: 27) | (SEQ ID NO: 28) | |
| hPMSR2 | 5′-ggcgcaaccaaagcaagag-3′ | 5′-actgcgttttttccgaacg-3′ | 221 |
| | (SEQ ID NO: 29) | (SEQ ID NO: 30) | |
| hPMSR3 | 5′-atgttggagaactacagcc-3′ | 5′-cactccatagtccttaagc-3′ | 278 |
| | (SEQ ID NO: 31) | (SEQ ID NO: 32) | |
| β-actin | 5′-gggaatgggtcagaaggac-3′ | 5′-tttcacggttggccttaggg-3′ | 209 |
| | (SEQ ID NO: 33) | (SEQ ID NO: 34) | |

Cell lines already determined to express PMSR2 and PMSR3 are used as positive controls while lines previously identified as PMSR null are used as negative controls. Samples are analyzed in duplicates to confirm reproducibility of expression.

To assess for microsatellite instability of lymphoma samples, DNAs are isolated from a panel of lymphomas as described above. DNAs will be isolated using the proteinase K digestion and phenol extraction procedure as described (Liu et al. (1996) Nature Med.2:169-174). Various amounts of test DNAs from lymphoma cells and HCT116 (a MMR defective human colon epithelial cell line) are used to determine the sensitivity of our microsatellite test. The D2S123, BAT26, and BAT40 alleles are known to be heterogeneous in HCT116 cells and are therefore used as a positive control for detection of MI. To measure for MI, DNAs are titrated by limiting dilution to determine the level of sensitivity for each marker set. DNAs are PCR amplified using the BAT26F: 5′-tgactacttttgacttcagcc-3′ (SEQ ID NO:35) and the BAT26R: 5′-aaccattcaacatttttaaccc-3′ (SEQ ID NO:36); BAT40F: 5′-attaacttcctacaccacaac-3′ (SEQ ID NO:37) and BAT40R: 5′-gtagagcaagaccaccttg-3′ (SEQ ID NO:38); and D2S123F: 5′-acattgctggaagttctggc-3′ (SEQ ID NO:39) and D2S123R: 5′-cctttctgacttggatacca-3′ (SEQ ID NO:40) primers in buffers as described (Nicolaides, N. C., et al. (1995) Genomics 30:195-206). Briefly, 1 pg to 100 ngs of DNA is amplified using the following conditions: 94° C. for 30 sec, 50-55° C. for 30 sec, 72° C. for 30 sec for 30 cycles. PCR reactions are then resolved on 8% denaturing polyacrylamide gels and visualized by autoradiography. Preliminary studies using these reagents and DNA extracted from paraffin-embedded tissues routinely find that 0.1 ng of genomic DNA is the limit of detection using our conditions.

Microsatellite stability may be measured in cells using twenty independent reactions of 0.01 ngs of DNA from the same clinical sample or cells by PCR. This concentration typically allows for the measurement of 1 genome equivalent per sample and allows for the detection of microsatellite alterations in clonal variants that have occurred during the growth of a particular cell line or tissue. Samples are scored MI+ if at least two samples of a particular marker are found to have PCR fragments that differ from the predominate allele size for a given sample. Statistical analysis is performed by comparing the number of MI+ cells expressing PMSR2 or PMSR3 with those not expressing either PMSR gene.

### Example 2

### Generation Of Polyclonal Antisera Specific For PMSR2 And PMSR3 For Immunostaining And Proof Of Concept At The Protein Level

The ability to produce antibodies that can specifically recognize PMSR2 or PMSR3 is of great utility for establishing methods for in situ analysis of tissues expressing these proteins as diagnostic markers. As demonstrated in FIG. 4, the generation of PMSR-specific peptides is used for tissue analysis to determine specific expression of a particular PMSR polypeptide. The immunoblot shown in FIG. 4 demonstrates the need for new antisera that allows for the specific detection of a PMSR protein without cross-reactivity to other PMS homologs. To generate PMSR specific antisera, we will synthesize 20 amino acid peptides and couple them to KLH immunogen for antisera production in rabbits. Peptides that are directed to the amino and carboxy termini of the hPMSR2 and hPMSR3 proteins may be generated by known methods. The amino acid sequences of the peptides to be synthesized are provided in Table 2. All peptides are directed to the first or last 20 amino acid residues of the encoded polypeptide (Nicolaides, N. C. et al. (1998) Mol. Cell. Biol. 18:1635-1641), except for the N-terminal hPMSR3 peptide which contains amino acids 5 to 26 to avoid multiple cysteine and tryptophan residues which have posed solubility problems for our group in the past.

TABLE 2

Peptides for PMSR2 and PMSR3 specific antisera

| Protein | N-terminal peptide | C-terminal peptide |
|---|---|---|
| hPMSR2 | MAQPKQERVARARHQRSETA | LEDNVITVFSSVKNGPGSSR |
| | (SEQ ID NO:41) | (SEQ ID NO:42) |
| hPMSR3 | RPRLGRRCMVSPRARAPREQ | GVEEENFEGLISFSSETSHM |
| | (SEQ ID NO:43) | (SEQ ID NO:44) |

The peptides produced are purified and analyzed by Mass Spectroscopy and HPLC analysis. 3 mgs of immunopure peptide are conjugated to keyhole limpet haemocyanin (KLH) carrier using a water-soluble carbodiimide, which eliminates the need for a cysteine residue in the sequence. The remaining peptide material is used for antisera analysis by ELISA and western blot. After conjugation, the KLH-linked peptide is resuspended in Freund's adjuvant and is ready for immunization.

Rabbits are immunized against each peptide using the following protocol. At Day 0, a prebleed will be taken from each host rabbit. Antigen is administered to rabbits by an injection of a solution containing adjuvant on a weekly schedule with three scheduled bleeds at day 49, 63, and 77, where a 20 ml sample of serum is collected and analyzed. Bleeds will be analyzed for antisera directed against immunizing peptides for PMSR2 and PMSR3 by Enzyme Linked Immuno-Sorbant Assay (ELISA) and western blots.

ELISA assays are performed to test antibody titer in unpurified bleeds to measure for antibody reactivity to native peptides described above. Briefly, 96 well plates are coated with 50 uls of a 1 ug/ml solution containing each peptide for 4 hours at 4° C. Wells containing each peptide are probed by each antiserum to measure for background and antibody specificity. Plates are washed 3 times in calcium and magnesium free phosphate buffered saline solution (PBS^{-/-}) and blocked in 100 uls of PBS^{-/-} with 5% dry milk for 1 hour at room temperature. After blocking, wells are rinsed and incubated with 100 uls of a PBS solution containing a 1:5 dilution of preimmune serum or respective bleeds from each rabbit for 2 hours. Plates are then washed 3 times with PBS^{-/-} and incubated for 1 hour at room temperature with 50 uls of a PBS^{-/-} solution containing 1:3000 dilution of a sheep anti-rabbit horseradish peroxidase (HRP) conjugated secondary antibody. Plates are then washed 3 times with PBS^{-/-} and incubated with 50 uls of TMB-HRP substrate (BioRad) for 15 minutes at room temperature to detect antibody titers. Reactions are stopped by adding 50 uls of 500 mM sodium bicarbonate and analyzed by OD at 415 nm using a BioRad plate reader. Samples are determined to be positive if an enhanced signal over background (preimmune serum and/or negative control peptides) are observed.

Western blot are also performed using antisera generated above as a probe to demonstrate the ability of antisera to recognize the expected molecular weight protein in whole cell extracts. First, unconjugated peptides are tested for antibody reactivity. The peptides listed in Table 2 are added to 20 μs of 2×SDS lysis buffer (60 mM Tris, pH 6.8/2% SDS/0.1 M 2-mercaptoethanol/0.1% bromophenol blue) and boiled for 2 min. Twenty microliters of each sample is then electrophoresed in 18% Tris-glycine SDS/PAGE gels for 10 minutes and electroblotted onto Immobilon-P (Millipore) membrane in transfer buffer (48 mM Tris/40 mM glycine/0.0375% SDS/20% methanol) for 20 minutes to maximize peptide binding. Filters are blocked overnight in blocking buffer (TBS, 0.05% Tween-20/5% powdered milk). Filters are probed with different prebleeds and antiserum from each rabbit followed by a secondary horseradish peroxidase conjugated anti-rabbit (Pierce) and prepared for chemiluminescence. Samples are deemed positive if the appropriate antisera reacts with the corresponding peptide antigen while no reaction is observed in negative or peptide control lanes. Samples are also deemed positive if no reaction is observed using preimmune serum.

The activity of positive antisera as described above is analyzed using whole cell lysates in western blot using extracts from cells previously identified to express PMSR2 and PMSR3 at the RNA and/or in the case of PMSR2, at the protein level (which is recognized by anti-PMS2 antisera, see FIG. 4). Fifty thousand cells are centrifuged and directly lysed in 25 μl of 2×sample buffer and boiled for 5 minutes. Samples are loaded on 4-20% Tris-glycine gels and electroblotted as described above except electrophoresis and transfer time is 1 hour. Filters are probed with various antisera and bleed lots and detected as above. Antisera are deemed positive if immunoreactions are observed in PMSR positive lines but are absent in PMSR negative cell lines. Positive reactions will be further confirmed for specificity by monitoring for endogenous PMS2 cross-reactivity as seen in FIG. 4 as well as competition using various peptides to monitor for binding. If background is observed in any antiserum, reaction conditions are altered by changing blocking buffers, washing stringencies, and dilution of antisera, parameters that have been routinely modified by our group for successful antibody probing.

PMSR specific antiserum may be purified using Pierce Ig purification kits, for example, that are able to purify total immunoglobulin to >95% purity. Antibody totals are quantitated by spectrophotometry, resuspended at a concentration of 1 mg/ml in PBS containing sodium azide as preservative. Antisera are re-tested for activity in western blot using 1:10, 1:100, and 1:1000 dilution to determine optimal concentration of pure materials. Purified antisera may then be used for immunohistological analysis of tissue blocks as described below.

If PMSR raised antisera are unable to detect the target protein in whole cell extracts then the antibody will be affinity-purified by linking the corresponding peptide to cyanogen bromide-activated agarose beads following the manufacturer's protocol (Pierce). Total antiserum will be incubated with affinity resin for 2 hours on a rotator wheel, washed in PBS buffer, followed by centrifugation for 5 cycles. Antibody is liberated from resin by incubation in acidic glycine buffer. Free antibody is added to neutralizing buffer in 1M Tris pH, 8.0. Antibody is then re-tested as described above.

### Example 3

### Analysis Of Other Tumor Sources For PMSR2 And PMSR3 Expression

A preliminary analysis of PMSR2 and PMSR3 expression was performed using RNAs from primary tissues as well as on a subset of colorectal tumor tissues and cell lines. A more extensive survey of other tissue types for PMSR2 and PMSR3 expression may be performed in light of the wide distribution of MI tumors that lack detectable mutations in the previously identified MMR genes (Xu, L. et al. (2001) Int. J. Cancer 91:200-204). Samples may be tested using tissue panels purchased from a supplier such as the NCI Tissue Array Research Program (TARP) sponsored by the Cooperative Human Tissue Network. Microarrays are screened with hPMSR2 and hPMSR3 antisera to monitor for expression in neoplastic specimens.

Immunohistochemistry of slides are performed using a standard protocol as described (Grasso, L. et al. (1998) J. Biol. Chem. 273:24016-24024). Briefly, paraffin embedded sections are incubated in xylene for 10 minutes each, followed by 2 minutes incubation in 100% ethanol. Next, samples are hydrated by placing them in 95%, 70%, 50%, 30% ethanol for 2 minutes each. Hydrated samples are then incubated for 30 minutes in 0.3% hydrogen peroxide in methanol to block endogenous peroxidase activity. Slides are washed in a chamber of running water for 20 minutes and placed in 0.25 M Tris-HCl pH 7.5 buffer. For immunostaining, slides are blocked with 10% goat serum in PBS for 20 minutes at room temperature in a humidified chamber followed by a final wash in PBS buffer. Antibody is diluted 1:20 in reaction buffer containing 0.25 M Tris-HCl pH 7.5; 0.5% BSA and 2% fetal calf serum and added onto the slide surface with enough volume to flood the tissue area. Slides are incubated at room temperature for 4 hours and washed in PBS for 5 minutes, blocked in reaction buffer for 5 minutes and probed with a secondary anti-rabbit HRP conjugated antibody diluted 1:200 in reaction buffer for 30 minutes in a humidified chamber. After secondary staining, slides are washed for 5 minutes in buffer as before. Sections are visualized by peroxidase staining using the Vectastain kit (Amersham) following the manufacturer's instructions. Reactions are stopped by rinsing in water after a uniform brown color becomes visible on the section. Reactions are carried out using antibodies with or without immunizing peptide as competitor to monitor for specific binding. Slides are examined via microscopy and scored positive in samples where internal staining is observed when the appropriate antibody is incubated alone or in the presence of nonsense peptide competitor but negative when antibody is incubated with blocking peptide. Samples will be repeated to confirm reproducibility.

### Example 4

### Generation Of Inducible MMR Dominant Negative Allele Vectors And Yeast Cells Harboring The Expression Vectors

Yeast expression constructs were prepared to determine if the human PMS2 related gene (hPMSR2) (Nicolaides, N. C. et al. (1995) Genomics 30(2):195-206) and the human PMS 134 gene (Nicolaides N. C. et al. (1998) Mol. Cell. Biol. 18:1635-1641) are capable of inactivating the yeast MMR activity and thereby increase the overall frequency of genomic hypermutation, a consequence of which is the generation of variant sib cells with novel output traits following host selection. For these studies, a plasmid encoding the hPMS 134 cDNA was altered by polymerase chain reaction (PCR). The 5′ oligonucleotide has the following structure: 5′-ACG CAT ATG GAG CGA GCT GAG AGC TCG AGT-3′ (SEQ ID NO:45) that includes the NdeI restriction site CAT ATG. The 3′-oligonucleotide has the following structure: 5′-GAA TTC TTA TCA CGT AGA ATC GAG ACC GAG GAG AGG GTT AGG GAT AGG CTT ACC AGT TCC AAC CTT CGC CGA TGC-3′ (SEQ ID NO:46) that includes an EcoRI site GAA TTC and the 14 amino acid epitope for the V5 antibody. The oligonucleotides were used for PCR under standard conditions that included 25 cycles of PCR (95° C. for 1 minute, 55° C. for 1 minute, 72° C. for 1.5 minutes for 25 cycles followed by 3 minutes at 72° C.).

The PCR fragment was purified by gel electrophoresis and cloned into pTA2.1 (Invitrogen) by standard cloning methods (Sambrook et al. MOLECULAR CLONING: A LABORATORY MANUAL, Third Edition, 2001), creating the plasmid pTA2.1-hPMS134. pTA2.1-hPMS 134 was digested with the restriction enzyme EcoRI to release the insert which was cloned into EcoRI restriction site of pPIC3.5K (Invitrogen). The following strategy, similar to that described above to clone human PMS134, was used to construct an expression vector for the human related gene PMSR2. First, the hPMSR2 fragment was amplified by PCR to introduce two restriction sites, an NdeI restriction site at the 5′end and an EcoRI site at the 3′-end of the fragment. The 5′-oligonucleotide that was used for PCR has the following structure: 5′-ACG CAT ATG TGT CCT TGG CGG CCT AGA-3′ (SEQ ID NO:47) that includes the NdeI restriction site CAT ATG. The 3′-oligonucleotide used for PCR has the following structure: 5′-GAA TTC TTA TTA CGT AGA ATC GAG ACC GAG GAG AGG GTT AGG GAT AGG CTT ACC CAT GTG TGA TGT TTC AGA GCT-3′ (SEQ ID NO:48) that includes an EcoRI site GAA TTC and the V5 epitope to allow for antibody detection. The plasmid that contained human PMSR3 in pBluescript SK (Nicolaides N. C. et al. (1995) Genomics 30(2):195-206) was used as the PCR target with the hPMS2-specific oligonucleotides above. Following 25 cycles of PCR (95° C. for 1 minute, 55° C. for 1 minute, 72° C. for 1.5 minutes for 25 cycles followed by 3 minutes at 72° C.). The PCR fragment was purified by gel electrophoresis and cloned into pTA2.1 (Invitrogen) by standard cloning methods (Sambrook et al., MOLECULAR CLONING: A LABORATORY MANUAL, Third Edition, 2001), creating the plasmid pTA2.1-hR2. pTA2.1-hR2 was next digested with the restriction enzyme EcoRI to release the insert (there are two EcoRI restriction sites in the multiple cloning site of pTA2.1 that flank the insert) and the inserted into the yeast expression vector pPIC3.5K (Invitrogen).

Pichia pastoris yeast cells were transformed with pPIC3.5K vector, pPIC3.5K-PMS134, and pPIC3.5K-hR2 as follows. First, 5 ml of YPD (1% yeast extract, 2% bacto-peptone, 1% dextrose) medium was inoculated with a single colony from a YPD plate (same as YPD liquid but add 2% Difco agar to plate) and incubated with shaking overnight at 30° C. The overnight culture was used to inoculate 500 ml of YPD medium (200 ul of overnight culture) and the culture incubated at 30° C. until the optical density at 600 nm reached 1.3 to 1.5. The cells were then spun down (4000×g for 10 minutes), and then washed 2 times in sterile water (one volume each time), then the cells suspended in 20 ml of 1 M sorbitol. The sorbitol/cell suspension was spun down (4,000×g for 10 minutes) and suspended in 1 ml of 1 M sorbitol. 80 ul of the cell suspension was mixed with 5 to 10 ug of linearized plasmid DNA and placed in a 0.2 cm cuvette, pulsed length 5 to 10 milliseconds at field strength of 7,500 V/cm. Next, the cells are diluted in 1 ml of 1 M sorbitol and transferred to a 15 ml tube and incubated at 30° C. for 1 to 2 hours without shaking. Next, the cells are spun out (4,000×G for 10 minutes) and suspended in 100 ul of sterile water, and 50 ul/plate spread onto the appropriate selective medium plate. The plates are incubated for 2 to 3 days at 30° C. and colonies patched out onto YPD plates for further testing.

### Example 5

### Generation Of Hypermutable Yeast With Inducible Dominant Negative Alleles Of Mismatch Repair Genes

Yeast clones expressing human PMS2 homologue PMS-R2 or empty vector were grown in BMG (100 mM potassium phosphate, pH 6.0, 1.34% YNB (yeast nitrogen base), 4×10−5% biotin, 1% glycerol) liquid culture for 24 hr at 30° C. The next day, cultures were diluted 1:100 in MM medium (1.34% YNB, 4×10−5% biotin, 0.5% methanol) and incubated at 30° C. with shaking. Cells were removed for mutant selection at 24 and 48 hours post methanol induction as described below (see Example 6).

### Example 6

### Dominant Negative MMR Genes Can Produce New Genetic Variants And Commercially Viable Output Traits In Yeast

The ability to express MMR genes in yeast, as presented in Example 5, demonstrates the ability to generate genetic alterations and new phenotypes in yeast expressing dominant negative MMR genes. In this example we teach the utility of this method to create eukaryotic strains with commercially relevant output traits.

### Generation of Uracil Dependent Yeast Strain

One example of utility is the generation of a yeast strain that is mutant for a particular metabolic product, such as an amino acid or nucleotide. Engineering such a yeast strain will allow for recombinant manipulation of the yeast strain for the introduction of genes for scalable process of recombinant manufacturing. In order to demonstrate that MMR can be manipulated in yeast to generate mutants that lack the ability to produce specific molecular building blocks, the following experiment was performed. Yeast cells that express a methanol inducible human PMS2 homologue, hPMS2-R2 (as described in Example 4 above), were grown in BMY medium overnight then diluted 1:100 and transferred to MM medium, which results in activation of the AOX promoter and production of the hPMS2-R2 MMR gene that is resident within the yeast cell. Control cells were treated the same manner; these cells contain the pPIC3.5 vector in yeast and lack an insert. Cells were induced for 24 and 48 hours and then selected for uracil requiring mutations as follows. The cells were plated to 5-FOA medium (Boeke, J. D. et al. (1984) Mol. Gen. Genet. 197:345-346). The plates are made as follows: (2×concentrate (filter sterilize): yeast nitrogen base 7 grams; 5-fluoro-orotic acid 1 gram; uracil 50 milligrams; glucose 20 grams; water to 500 ml; Add to 500 ml 4% agar (autoclaved) and pour plates. Cells are plated on 5-FOA plates (0, 24 and 48 hour time points) and incubated at 30° C. for between 3 and 5 days. Data from a typical experiment is shown in Table 3. No uracil requiring clones were observed in the un-induced or induced culture in yeast cells that harbor the “empty” vector whereas those cells that harbor the MMR gene hPMS2-R2 have clones that are capable of growth on the selection medium. Note that the uninduced culture of hPMS2-R2 does not have any colonies that are resistant to 5-FOA, demonstrating that the gene must be induced for the novel phenotype to be generated.

It has been demonstrated that the mutagens (such as ethyl methyl sulfonate result in a low number of ura mutants and that the spontaneous mutation rate for generating this class of mutants is low (Boeke, J. D. et al. (1984) Mol. Gen. Genet. 197:345-346).

TABLE 3
Generation of uracil requiring mutant Pichia pastoris yeast cells.

| | | | | Frequency |
|---|---|---|---|---|
| Strain | Seeded | ura− | URA+ | (ura− cells) |
| Wt | 100,000 | 0 | ˜100,000 | 0 |
| Empty | 100,000 | 0 | ˜100,000 | 0 |
| pMOR^{ye-1#} | 100,000 | 14 | ˜100,000 | 1/7,142 |
| pMOR^{ye2@} | 100,000 | 123 | ˜100,000 | 1/813 |
| Wt | 100,000 | 1-0.1 | 100,000 | 1/10^{5-6*} |
| Mutagen | 100,000 | 10 | 100,000 | 1/10,000 |

^{#}Represents at 24 hour methanol induction and
^{@}a 48 hour induction.
For comparison a wild type yeast cell treated/un-treated is shown (Galli, A. and R. H. Schiestl, (1999) Mutat. Res. 429(1):13-26).

### Generation Of Heat-resistant Producer Strains

One example of commercial utility is the generation of heat-resistant recombinant protein producer strains. In the scalable process of recombinant manufacturing, large-scale fermentation of both prokaryotes and eukaryotes results in the generation of excessive heat within the culture. This heat must be dissipated by physical means such as using cooling jackets that surround the culture while it is actively growing and producing product. Production of a yeast strain that can resist high temperature growth effectively would be advantageous for large-scale recombinant manufacturing processes. To this end, the yeast strain as described in Example 5 can be grown in the presence of methanol to induce the dominant negative MMR gene and the cells grown for various times (e.g. 12, 24, 36 and 48 hours) then put on plates and incubated at elevated temperatures to select for mutants that resist high temperature growth (e.g., 37° C. or 42° C.). These strains would be useful for fermentation development and scale-up of processes and should result in a decrease in manufacturing costs due to the need to cool the fermentation less often.

### Generation Of High Recombinant Protein Producer Strains And Strains With Less Endogenous Protease Activity

Yeast is a valuable recombinant-manufacturing organism since it is a single celled organism that is inexpensive to grow and easily lends itself to fermentation at scale. Further more, many eukaryotic proteins that are incapable of folding effectively when expressed in Escherichia coli systems fold with the proper conformation in yeast and are structurally identical to their mammalian counterparts. There are several inherent limitations of many proteins that are expressed in yeast including over and/or inappropriate glycosylation of the recombinant protein, proteolysis by endogenous yeast enzymes and insufficient secretion of recombinant protein from the inside of the yeast cell to the medium (which facilitates purification). To generate yeast cells that with this ability to over-secrete proteins, or with less endogenous protease activity and or less hyper-glycosylation activity yeast cells as described in Example 4 can be grown with methanol for 12, 24, 36 and 48 hours and yeast cells selected for the ability to over-secrete the protein or interest, under-glycosylate it or a cell with attenuated of no protease activity. Such a strain will be useful for recombinant manufacturing or other commercial purposes and can be combined with the heat resistant strain outlined above.

For example, a mutant yeast cell that is resistant to high temperature growth and can secrete large amounts of protein into the medium would result.

Similar results were observed with other dominant negative mutants such as the PMSR2, PMSR3, and the human MLH1 proteins.

### Example 7

### Mutations Generated In The Host Genome Of Yeast By Defective MMR Are Genetically Stable

As described in Example 6 manipulation of the MMR pathway in yeast results in alterations within the host genome and the ability to select for a novel output traits, for example the ability of a yeast cell to require a specific nutrient. It is important that the mutations introduced by the MMR pathway is genetically stable and passed to daughter cells reproducibly once the wild type MMR pathway is re-established. To determine the genetic stability of mutations introduced into the yeast genome the following experiment was performed. Five independent colonies from pPIC3.5KhPMS2-R2 that are ura-, five wild type control cells (URA+) and five pPIC3.5K transformed cells (“empty vector”) were grown overnight from an isolated colony in 5 ml of YPD (1% yeast extract, 2% bacto-peptone and 1% dextrose) at 30° C. with shaking. The YPD medium contains all the nutrients necessary for yeast to grow, including uracil. Next, 1 pL of the overnight culture, which was at an optical density (OD) as measured at 600 nM of >3.0, was diluted to an OD600 of 0.01 in YPD and the culture incubated with shaking at 30° C. for an additional 24 hours. This process was repeated 3 more times for a total of 5 overnight incubations. This is the equivalent of greater than 100 generations of doubling (from the initial colony on the plate to the end of the last overnight incubation. Cells (five independent colonies that are ura and five that were wild type were then plated onto YPD plates at a cell density of 300 to 1,000 cells/plate and incubated for two days at 30° C. The cells from these plates were replica plated to the following plates and scored for growth following three days incubation at 30° C.; Synthetic Complete (SC) SC-ura (1.34% yeast nitrogen base and ammonium sulfate; 4×10−5% biotin; supplemented with all amino acids, NO supplemental uracil; 2% dextrose and 2% agar); SC +URA (same as SC-ura but supplement plate with 50 mg uracil/liter medium), and YPD plates. They were replica plated in the following order-SC-ura, SC complete, YPD. If the novel output trait that is resident within the yeast genome that was generated by expression of the mutant MMR (in this example the human homologue of PMS2, hPMS2-R2) is unstable, the uracil dependent cells should “revert” back a uracil independent phenotype. If the phenotype is stable, growth of the mutant cells under non-selective conditions should result in yeast cells that maintain their viability dependence on exogenous supplementation with uracil. As can be seen in the data presented in Table 4, the uracil dependent phenotype is stable when the yeast cells are grown under non-selective conditions, demonstrating that the MMR-generated phenotype derived from mutation in one of the uracil biosynthetic pathway genes is stable genetically.

TABLE 4

| Strain | Seeded | −ura | +URA | YPD |
|---|---|---|---|---|
| Wt | 650 | 650 | 650 | 650 |
| Empty | 560 | 560 | 560 | 560 |
| pMOR^{ye-1#} | 730 | 0 | 730 | 730 |

These data demonstrate the utility of employing an inducible expression system and a dominant negative MMR gene in a eukaryotic system to generate genetically altered strains. The strain developed in this example, a yeast strain that now requires addition of uracil for growth, is potentially useful as a strain for recombinant manufacturing; by constructing an expression vector that harbors the wild type URA3 gene on either an integration plasmid or an extra-chromosomal vector it is now possible to transform and create novel cells expressing the a protein of interest. It is also possible to modify other resident genes in yeast cells and select for mutations in genes that that give other useful phenotypes, such as the ability to carry out a novel biotransformation. Furthermore, it is possible to express a gene extra-chromosomally in a yeast cell that has altered MMR activity as described above and select for mutations in the extra-chromosomal gene. Therefore, in a similar manner to that described above the mutant yeast cell can be put under specific selective pressure and a novel protein with commercially important biochemical attributes selected.

These examples are meant only as illustrations and are not meant to limit the scope of the present invention.

Finally, as described above once a mutation has been introduced into the gene of interest the MMR activity is attenuated of completely abolished. The result is a yeast cell that harbors a stable mutation in the target gene(s) of interest.

### Example 8

### Enhanced Generation Of MMR-Defective Yeast And Chemical Mutagens For The Generation Of New Output Traits

It has been previously documented that MMR deficiency yields to increased mutation frequency and increased resistance to toxic effects of chemical mutagens (CM) and their respective analogues such as but not limited to those as: ethidium bromide, EMS, MNNG, MNU, Tamoxifen, 8-Hydroxyguanine, as well as others listed but not limited to in publications by: Khromov-Borisov, N. N., et al. (1999) Mutat. Res. 430:55-74; Ohe, T. et al. (1999) Mutat. Res. 429:189-199; Hour, T. C. et al. (1999) Food Chem. Toxicol. 37:569-579; Hrelia, P. et al. (1999) Chem. Biol. Interact. 118:99-111; Garganta, F. et al. (1999) Environ. Mol. Mutagen. 33:75-85; Ukawa-Ishikawa S. et al. (1998) Mutat. Res. 412:99-107; www.ehs.utah.edu/ohh/mutagens; Marcelino, L. A. et al (1998) Cancer Res. 58(13):2857-2862; Koi, M. et al. (1994) Cancer Res. 54:4308-4312. Mismatch repair provokes chromosome aberrations in hamster cells treated with methylating agents or 6thioguanine, but not with ethylating agents. To demonstrate the ability of CMs to increase the mutation frequency in MMR defective yeast cells, we would predict that exposure of yeast cells to CMs in the presence or absence of methanol (which induces the expression of the resident human homologue to PMS2, hPMS2-R2) will result in an augmentation of mutations within the yeast cell.

Yeast cells that express hPMS2-R2 (induced or un-induced) and empty vector control cells are grown as described in Examples 5 and 6) and for 24 hours and diluted into MM medium as described above. Next, the cells in MM are incubated either with or without increasing amounts of ethyl methane sulfonate (EMS) from 0, 1, 10, 50, 100, and 200 pM. 10 zip aliquots of culture (diluted in 300 ul MM) and incubated for 30 minutes, 60 minutes, and 120 minutes followed by plating cells onto 5-FOA plates as described in Example 3 above. Mutants are selected and scored as above. We would predict that there will be an increase in the frequency of ura mutants in the PMS2-R2 cultures that are induced with methanol as compared to the uninduced parental or wild type strain. In a further extension of this example, human PMS2-R2 harboring cells will be induced for 24 and 48 hours then mutagenized with EMS. This will allow the MMR gene to be fully active and expressed at high levels, thereby resulting in an increase in the number of ura mutants obtained. We would predict that there will be no change in the number of ura mutants obtained in the uninduced parental control or the wild type “empty vector” cells. This example demonstrates the use of employing a regulated dominant negative MMR system plus chemical mutagens to produce enhanced numbers of genetically altered yeast strains that can be selected for new output traits. This method is useful for generating such organisms for commercial applications such as but not limited to recombinant manufacturing, biotransformation, and altered biochemicals with enhanced activities. It is also useful to obtain alterations of protein activity from ectopically expressed proteins harbored on extra-chromosomal expression vectors similar to those described in Example 4 above.

### Examples Of MMR Genes And Encoded Polypeptides

Yeast MLH1 cDNA (accession number U07187) (SEQ ID NO:1); yeast MLH1 protein (accession number U07187) (SEQ ID NO:2); mouse PMS2 protein (SEQ ID NO:3); mouse PMS2 cDNA (SEQ ID NO:4); human PMS2 protein (SEQ ID NO:5); human PMS2 cDNA (SEQ ID NO:6); human PMS1 protein (SEQ ID NO:7); human PMS1 cDNA (SEQ ID NO:8); human MSH2 protein (SEQ ID NO:9); human MSH2 cDNA (SEQ ID NO:10); human MLHI protein (SEQ ID NO:11); human MLH1 cDNA (SEQ ID NO:12); hPMS2-134 protein (SEQ ID NO:13); hPMS2-134 cDNA (SEQ ID NO:14); hMSH6 (human protein) (accession number U28946 (SEQ ID NO:15); hMSH6 (human cDNA) (accession number U28946) (SEQ ID NO:16); hPMSR2 (human cDNA) (accession number U38964) (SEQ ID NO:17); hPMSR2 (human protein) (accession number U38964) (SEQ ID NO:18); HPMSR3 (human cDNA) (accession number NM_{—}005395.1) (SEQ ID NO:19); hPMSR3 (human protein) (accession number U38979.1) (SEQ ID NO:20); hPMSR6 (human cDNA) (accession number U38980.1) (SEQ ID NO:21); hPMSR6 (human protein) (accession number U38980.1) (SEQ ID NO:22).

What is claimed is:

1. A method of making a cell hypermutable comprising introducing into said cell a PMS2 homolog comprising a nucleotide sequence encoding a polypeptide comprising the amino acid sequence of SEQ ID NO:23, thereby making a hypermutable cell, wherein said PMS2 homolog is other than PMSR2 and PMSR3.

2. The method of claim 1 wherein said polypeptide comprises the amino acid sequence of SEQ ID NO:24.

4. The method of claim 1 wherein said polypeptide comprises the amino acid sequence of SEQ ID NO:22.

5. The method of claim 1 wherein said PMS2 homolog encodes a protein having an ATPase domain.

6. The method of claim 1 wherein said cell is a eukaryotic cell.

7. The method of claim 1 wherein said cell is a prokaryotic cell.

8. The method of claim 6 wherein said cell is a mammalian cell.

9. The method of claim 8 wherein said cell is a human cell.

10. The method of claim 1 further comprising contacting said cell with a mutagen.

11. The method of claim 1 or 10 further comprising screening said cell for a mutation in a gene of interest.

12. The method of claim 11 wherein said screening is performed on the nucleic acid of said hypermutable cell.

13. The method of claim 11 wherein said screening is performed on the protein of said hypermutable cell.

14. The method of claim 11 wherein said screening is performed by examining the phenotype of said hypermutable cell.

15. The method of claim 11 further comprising restoring genetic stability of said hypermutable cell.

16. A method of making a mutation in a gene of interest comprising introducing into a cell containing a gene of interest a PMS2 homolog comprising a nucleotide sequence encoding a polypeptide comprising the amino acid sequence of SEQ ID NO:23, thereby making said cell hypermutable, and selecting a mutant cell comprising a mutation in said gene of interest.

17. The method of claim 16 wherein said polypeptide comprises the amino acid sequence of SEQ ID NO:24.

18. The method of claim 16 wherein said polypeptide comprises the amino acid sequence of SEQ ID NO:22.

19. The method of claim 16 wherein said PMS2 homolog encodes a protein having an ATPase domain.

20. The method of claim 16 wherein said cell is a eukaryotic cell.

21. The method of claim 16 wherein said cell is a prokaryotic cell.

22. The method of claim 20 wherein said cell is a mammalian cell.

23. The method of claim 22 wherein said cell is a human cell.

24. The method of claim 16 further comprising contacting said cell with a mutagen.

25. The method of claim 16 or 24 further comprising restoring genetic stability of said mutant cell.

26. A method of generating a library of mutant genes in a cell type comprising introducing into said cell type a PMS2 homolog comprising a nucleotide sequence encoding a polypeptide comprising the amino acid sequence of SEQ ID NO:23, thereby making hypermutable cells, wherein said PMS2 homolog is other than PMSR2 and PMSR3, incubating said hypermutable cells type to allow mutations to accumulate, extracting nucleic acid from said hypermutable cells and creating a nucleic acid library.

27. The method of claim 26 wherein said polypeptide comprises the amino acid sequence of SEQ ID NO:24.

28. The method of claim 27 wherein said library is a cDNA library.

29. The method of claim 27 wherein said library is a genomic library.

30. A method of assaying cells to detect neoplasia comprising contacting said sample with a nucleotide sequence encoding the amino acid sequence of SEQ ID NO:23 to detect expression of a polynucleotide encoding a PMS2 homolog comprising the amino acid sequence of SEQ ID NO:23, wherein expression of said PMS2 homolog is associated with neoplasia.

31. The method of claim 30, wherein the detecting comprises a Northern blot analysis.

32. The method of claim 30, wherein the detecting comprises PCR.

33. The method of claim 30, wherein detecting comprises RT-PCR analysis.

34. A method of assaying cells to detect neoplasia comprising contacting said sample with an antibody directed against a PMS2 homolog or peptide fragments thereof; and detecting the presence of an antibody-complex formed with the PMS2 homolog or peptide fragment thereof, thereby detecting the presence of said PMS2 homolog in said sample, wherein the presence of said PMS2 homolog is associated with neoplasia.

35. The method of claim 34, wherein the detecting comprises an immunoassay selected from the group consisting of a radioimmunoassay, a Western blot assay, an immunofluorescent assay, an enzyme-linked immunosorbent assay, and a chemiluminescent assay.

36. A method of treating a patient with cancer comprising identifying a patient with a PMS2 homolog-associated neoplasm, administering to said patient an inhibitor of expression of said PMS2 homolog wherein said inhibitor suppresses expression of said PMS2 homolog in said PMS2 homolog associated neoplasm.

37. The method of claim 36 wherein said PMS2 homolog associated neoplasm is a lymphoma.

38. The method of claim 36 wherein said inhibitor of said PMS2 homolog is an antisense nucleic acid directed against a polynucleotide encoding said PMS2 homolog.

39. The method of claim 36 wherein said inhibitor of said PMS2 homolog is a ribozyme.

40. The method of claim 36 wherein said inhibitor is a ATPase analog that specifically binds to said PMS2 homolog.
